(19) United States
(12) Patent Application Publication
(10) Pub. No.: US 2012/0203031 A1
(43) Pub. Date: Aug. 9, 2012

(54) Sustainable chemical process for reduction of nitro compounds (R-NO2) or nitroso compounds (R-NO) containing sulphonic or carboxylic group into corresponding amino compounds (R-NH2) with inherent recycle of all acidic streams generated in synthesis

(75) Inventors: Bhadresh K. Padia, Mumbai (IN); Nitesh H. Mehta, Mumbai (IN)

(73) Assignee: NEWREKA CHEMICALS PRIVATE LIMITED, Chembur. Mumbai (IN)

(21) Appl. No.: 13/502,526

(22) PCT Filed: Oct. 18, 2010
(86) PCT No.: PCT/IB2010/054698

§ 371 Date: Apr. 18, 2012

(30) Foreign Application Priority Data
Oct. 19, 2009 (IN) 1646/MUM/2009

(51) Int. Cl. C07C 303/22; C07C 227/04

(52) U.S. Cl. 562/60; 562/62; 562/58; 562/458; 562/453

(57) ABSTRACT

The process of the present invention creates a sustainable and closed water loop allowing inherent recycles of all liquid streams generated in the process. The liquid streams generated during the process of the invention are inherently recycled completely, making the process of the present invention a zero liquid discharge process which is environmentally friendly and sustainable. This invention further relates to a sustainable chemical process of reduction of R—NO₂ or R—NO into corresponding R—NH₂ that produces environmentally friendly R—NH₂ in good yields and selectivity with large of mother liquor recycle. The process has a wide scope in that it can be applied to a number of molecules.

### FIELD OF INVENTION

This invention relates to a process for the reduction in general and in particular to reduction of nitro (R—NO₂) or nitroso (R—NO) compounds containing sulphonic or carboxylic group into the corresponding amino compounds (R—NH2) with Isolation of amines and total recycle of acidic mother liquor.

### BACKGROUND OF INVENTION

Reduction, broadly defined as addition of hydrogen or removal of oxygen from any chemical, is one of the important chemical processes extensively applied in the manufacture of many molecules. Partial or complete reduction of functional groups such as nitro, nitroso, carbonyls, azides, nitriles, azo, and the like yields value added products.

Reduction of R—NO2/R—NO compounds into corresponding R—NH₂ finds applications in various groups of chemical including pharmaceuticals, dyes and pigments, agrochemicals, specialty chemicals, fine chemicals and explosives.

Many dyes, specialty fine chemicals have R—NH₂ as one of the building blocks and in most of the processes this important building block is obtained from the reduction of R—NO2/R—NO precursor. Some amino compounds like 4,4 Di amine diphenylamine 2-sulphonic acid (F.C Acid), meta phenylene di amine 4-sulphonic acid (MPDSA), 4-4 Di Amino Stilbene 2,2 Di Sulphonic Acid (DASDA), Di-amino benzoic acid (DABA), 5-amino salicylic acid, OPASA, ADAPSA, and 4-CAP acid are being currently used to manufacture intermediates for Dyestuffs & pigment and pharmaceutical industry.

Methods used for reduction of R—NO2 or R—NO into corresponding R—NH₂ can be broadly divided into three major categories: (a) chemical reduction (b) catalytic reduction, (c) electrochemical reduction.

Skipka, G. et. al., Ger. Offen. DE 2930754 (1981); Skalicky, P. et. al., Czech. CS 248864 B1 (1988), Laucoiner, M. et. al. in Appl. Catal., A, 172(1), 141-148 (1998) disclose methods of chemical reduction, which include metal and acid reduction such as Bechamp reduction, sulfide reduction, metal hydride reduction, hydrazine hydrate reduction and the like. Some of the drawbacks of these methods are that these processes require extreme pH conditions, handling of strong acids/strong alkalis, working in poisonous H₂S/SO₂ atmosphere, handling of pyrophoric and/or explosive material, and expensive material of construction (MOC) for the equipment.

Another drawback is that these methods generate large quantities of liquid waste which is difficult to recycle. These methods require environmentally unsustainable reaction conditions in terms of pH, temperature, concentration, reaction agents and medium, and so on, and leave an unsustainable impact on the environment, specifically on our water bodies.

Shimanzu, K. et. al. JP 9,132,536 (1997); Kuo, E. et. al. in Syn. Commun. 15(7), 599 (1985), disclose methods of catalytic reduction. The drawbacks of these methods, which normally use noble metal catalysts, are that they use highly inflammable hydrogen gas and require high pressure and/or high temperature. Further drawbacks of these methods are that they involve catalyst poisoning and regeneration, handling of pyrophoric catalysts, high cost due to use of pressure reactors and noble metal catalysts resulting in expensive, unsafe, and unsustainable, and inherently non-recyclable processes.

Gunawardena, N. E. et. al., Acta. Chem. Scand., Ser. B, B37 (6), 549-53 (1983); Starke, C. et. al. in Chem. Tech. (Leipzig), 35(9), 463-5 (1983) describe electrochemical reduction methods. These methods suffer from drawbacks such as poor conversion rates and low yields, and that these methods require large quantum of electricity, rendering these methods uneconomical and environmentally unsustainable.

Pelster-Heinrich FR 1481040 (1967), Hu, Zhangyun from Ranliao Gongye (2002), 39(4), 32-34, Luo, Junlong from Faming Zhuanli Shenqing Gongkai Shuomingshu (2009), CN 101362710 A 20090211, Wang, Zaijun; Shi, Yan from Faming Zhuanli Shenqing Gongkai Shuomingshu (2009), CN 101337915 A 20090107, Sun, Chunbao; Lin, Hai; Wang, Zuosheng; Song, Bo from Faming Zhuanli Shenqing Gongkai Shuomingshu (2006), CN 1807269 A 20060726, By Wang, Yong-guang from Anquan Yu Huanjing Gongcheng (2006), 13(1), 70-72, 76, Rao, R. Nageswara; Venkateswarlu, N.; Khalid, Sara; Narsimha, R.; Sridhar, S. from Journal of Chromatography, A (2006), 1113(1-2), 20-31, Chai, Li-min; Zhang, Feng-bao; Zhang, Guo-liang from Desalination (2005), 180(1-3), 157-162, Horsch, Philip; Speck, Andreas; Frimmel, Fritz H. from Water Research (2003), 37(11), 2748-2756, Rao, R. Nageswara; Venkateswarlu, N. from Process Biochemistry (Amsterdam, Netherlands) (2006), 41(5), 1097-1105, describes effluent treatment generated during the synthesis using various chemical &/or physical methods such as, electrolysis, chemical oxidation, reverse osmosis, vacuum distillation, ion exchange resin base separation and so forth. Inherently these methods are describes effluent treatment method which is end of pipe solution and not the recycle of mother liquor.

Furthermore, other drawback of all methods described above is that undesirable organic and inorganic side products are always formed as a result of these methods. The type of side products formed and their quantities vary from process to process and from molecule to molecule. This difficulty during recycle of acidic mother liquor results in generation of large quantities of liquid effluents.

Yet another drawback of the methods referred to above is that in some cases inorganic solid wastes are also generated. These solid wastes are contaminated with organic compounds like starting material, product and side products and pose a serious pollution problem. Normally these wastes are sticky solids and are tedious to handle and are difficult to dispose off, and are known in the industry as non-green solid wastes.

There are still further drawbacks of the above methods. Because of generation of large quantities of acidic liquid effluents and non-green solid wastes, above processes require a large facility for effluent treatment and disposal of solid wastes. These factors impose location related constraints making it mandatory for these processes to be carried out only in designated industrial areas, suitable for handling special chemicals.

Thus, these methods are non-green, unsustainable, uneconomical, and harm the environment to a very large extent, which has worldwide become a major concern.

Hence there is a need for providing a process of green reduction of R—NO₂ or R—NO compounds into corresponding amino compounds (R—NH₂) with inherent recycle of acidic mother liquor, thereby avoiding above mentioned disadvantages and drawbacks, and providing an environmentally sustainable and economical recycling solution.

### OBJECTS AND ADVANTAGES OF INVENTION

In order to overcome the various serious drawbacks of the existing methods, the inventors at ‘Newreka chemicals Pvt. Ltd.’ have developed a novel process using commercially available customized formulations such as G-Cat, R-Cat, for the reduction of R—NO₂ or R—NO into corresponding R—NH₂.

An object of the process of the present invention is to provide an environmentally friendly (green) process that overcomes the problem of generation of large quantities of acidic waste resulting from the conventional processes of reduction.

Another object of the present invention is to provide a process, wherein undesirable side reactions leading to organic and inorganic side products formation are substantially reduced by the virtue of chemo-selectivity and regio-selectivity which results in purer product formation.

An advantage of the present invention is that since both the number and quantity of organic and inorganic impurities are comparatively less, the possibility of build of side products in acidic mother liquor during recycle is less due to use of proprietary formulation G-Cat & R-Cat. This fact advantageously makes possible large number of mother liquor recycles in our process.

A further advantage of the method of present invention is that the product isolation processes disclosed herein ensure that the solid spent formed in the process of the present invention has surprisingly low levels of organic compounds and are thus green in nature. Consequently, the process described herein does not require any acidic liquid effluent treatment facility or elaborate solid waste disposal facility. A further advantage of the process of the present invention over the prior art is that the process is not constrained in respect of plant location, in that it doesn't necessarily have to be carried out in industrial areas.

A still further advantage of the present invention is that the inorganic by-product is non-sticky, which makes their handling easier and simpler than conventional processes.

Yet another advantage of the present invention is that the method disclosed herein not only is green and sustainable but the R—NH₂ produced by the process is also greener owing to the fact that they have fewer impurities. This makes downstream processing and application that involve these R—NH₂ highly recyclable.

A yet further advantage of the present invention is that the method disclosed herein is carried out at milder acid concentrations and at atmospheric pressure, which makes it safer.

Another advantage of the present invention is that the use of the proprietary reaction formulations developed by the inventors, namely G-Cat and R-Cat or any other similar formulations used in the process of this invention makes it possible to recycle the acidic process liquid streams completely.

A still further advantage of the process of the present invention is that its inherent thermodynamic conditions defined in terms of pressure, temperature, pH, concentrations of reaction components, and various reaction agents is close to respective conditions naturally occurring in the nature, thereby making the process of the invention benign and environmentally friendly.

There are several other key advantages of the process of the present invention. These relate to health and safety, process engineering, process economics. The process of the present invention is inherently safe due to the safe levels of the process parameters such as pressure, temperature, pH, concentrations of reaction components, and various reaction agents. This reduces the risk of injuries to the personnel and damage to the process plant.

The simplicity of the process also makes its engineering design simple. One other key advantage is that the plant and process breakdowns that could take place due to factors such as power failure, or uncontrolled fluctuations in the process parameters, do not affect the recyclability of the process. This leads to reduction in wastage on account of batch failures. The process therefore is able to avoid sudden shocks to the environment and sudden safety shocks to the plant and the personnel, ultimately leading to sustainable health of plant and personnel.

The process is carried out at such temperatures and pH values that it saves energy and therefore results in the process economy.

### SUMMARY OF THE INVENTION

The process of the present invention creates a sustainable and closed water loop allowing inherent recycles of all liquid streams generated in the process. Proprietary chemical agents are used in a novel manner which makes the process of the invention feasible. The liquid streams generated during the process of the invention are inherently recycled completely, making the process of the present invention a zero liquid discharge process which is green and sustainable.

This invention further relates to a sustainable chemical process of green reduction of R—NO₂ or R—NO into corresponding R—NH₂ that produces greener R—NH₂ in good yields and selectivity with large of mother liquor recycle.

The process has a wide scope in that it can be applied to a number of molecules.

### BRIEF DESCRIPTION OF FIGURES

FIG. 1 shows a green reaction sequence with complete and large number of acidic mother liquor recycles.

FIG. 2 shows green isolation sequence with complete and large number of mother liquor and washing streams.

FIG. 3 shows the schematic representation of complete process of the present invention.

FIG. 4 shows a simplified schematic relationship between individual cycles of the large number recycle loop.

FIG. 5 shows a schematic closed loop with large number of recycle of mother liquor in process of the invention

### DETAILED DESCRIPTION OF THE INVENTION

In order to aid the understanding of the process described herein several terms are explicitly defined.
Reaction medium (RM) is the solvent or water or a combination thereof used in the reaction.
Fresh reaction medium (FRM) is fresh water or fresh solvent or a combination thereof used in the reaction.
Solvent is any suitable solution that is water miscible, water immiscible, aromatic, and aliphatic or mixture thereof.
Reaction medium factor (RMF) is the ratio of the weight of FRM or RM with weight of R—NO₂ or R—NO used in the process.
Mother liquor (ML) is the liquid stream generated after performing a particular step. Mother liquor has been used as the RM at various stages of the process of the invention in its cycles following the first cycle.
Cooling curve (CC) is profile of temperature verses time.
G-CAT is the customized catalytic formulation has been used as the reducing agent.
R-CAT is the customized catalytic formulation has been used as the neutralizing agent.

The process of the present invention uses a proprietary reduction agent, G-CAT, which is a multifunctional, chemical reduction formulation mainly comprising of fine iron powder in the range of 50% (w/w) to 100% (w/w), preferable range being 75% (w/w) to 95% (w/w), tin powder and or zinc powder in the range of 0% (w/w) to 10% (w/w). The purity of all components is in the range of 50% (w/w) to 100% (w/w). It also contains electrolyte salt of various metals such as sodium, magnesium, calcium, iron, nickel, cobalt, tin, zinc, titanium, copper, manganese, and any other metals with multiple valancies in the range of 0% (w/w) to 50% (w/w), preferable range being 2.5% (w/w) to 25% (w/w). The purity of the salts is variable and in the range of 50% (w/w) to 100% (w/w). The G-CAT also contains customized grade of activated carbon in the range of 0% (w/w) to 5% (w/w); filter aid in the range of 0% (w/w) to 95% (w/w) and decolourizing agent in the range of 0% (w/w) to 5% (w/w). It also contains specialty additives like polyelectrolytes, anti foaming agents, dispersing agents, anti oxidants, emulsifying agents, mass transfer enhancing agents, anti caking agents, UV stabilizers, solubilising agents, and such other agents.

Another chemical formulations also used in the process of the present invention, namely R-Cat. Each of these formulations is a multifunctional recycle formulation mainly comprising fine iron powder in the range of 0% (w/w) to 95% (w/w), tin, copper, titanium, and zinc, or any combination thereof, depending on the R—NO₂ or R—NO to be reduced, in the range of 0% (w/w) to 10% (w/w). The purity of all components is in the range of 50% (w/w) to 100% (w/w).

R-Cat also contain electrolyte salt of various metals such as sodium, magnesium, calcium, iron, nickel, cobalt, tin, zinc, titanium, copper, manganese, and any other metals with multiple valency in the range of 0% (w/w) to 50% (w/w). The purity of the salts is variable and in the range of 50% (w/w) to 100% (w/w). The G-CAT also contains customized grade of activated carbon in the range of 0% (w/w) to 5% (w/w); filter aid in the range of 0%-95% and decolourizing agent in the range of 0% (w/w) to 5% (w/w).

R-Cat also contain hydroxides of calcium or alkali metals like magnesium, barium, sodium, potassium in the range of 0% (w/w) to 95% (w/w); customized grade of activated carbon in the range of 0.5% (w/w) to 5% (w/w); filter aid in the range of 5% (w/w) to 95% (w/w) and decolourizing agent in the range of 0.5% (w/w) to 5% (w/w) along with iron powder in the range of 5% (w/w) to 25% (w/w).

R-Cat also contain specialty additives like polyelectrolytes, anti foaming agents, dispersing agents, emulsifying agents, mass transfer enhancing agents, anti caking agents, UV stabilizers, solubilising agents, and anti oxidants, and other such agents.

R-Cat and G-Cat may contain additives like Hydrose, anti oxidants, crystallization agent etc to improve isolation, precipitation, crystallization and colour property. The present application also discloses a sustainable chemical process for green reduction of R—NO₂ or R—NO into corresponding R—NH₂ with inherent recycle of all acidic liquid streams generated in the same.

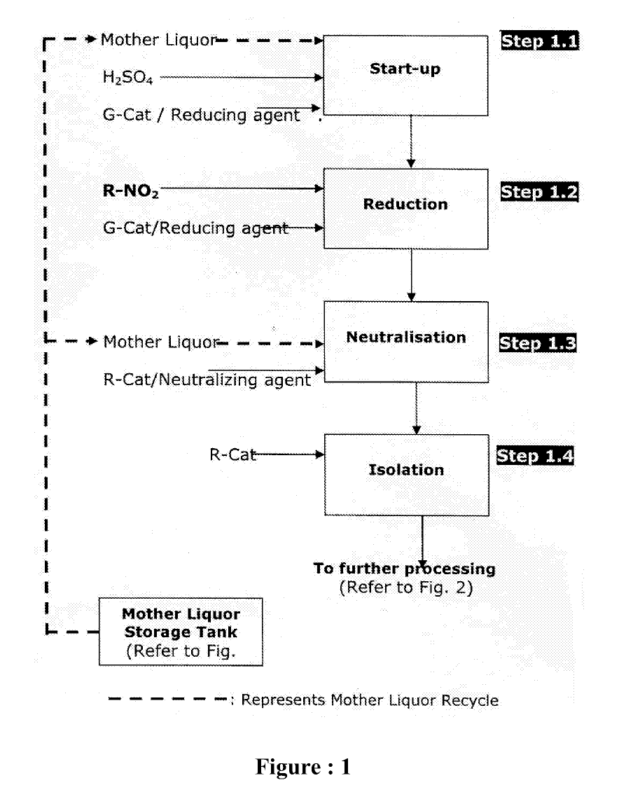

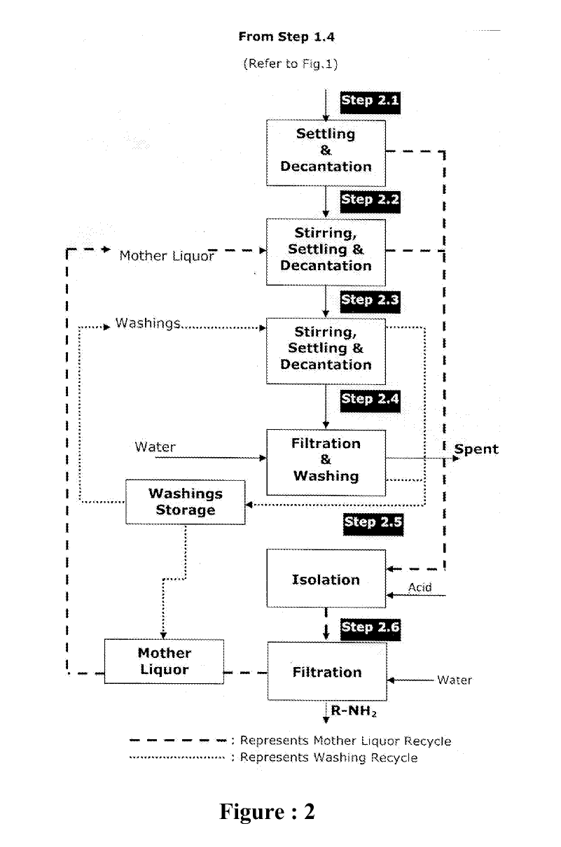

The chemical process of the present invention basically comprises inherent large number of recycles of processing the acidic mother liquor and all liquid streams generated during any of the cycles. Each cycle further comprises two sequences. The first sequence of typical cycle is represented in FIG. 1 and is termed as the green reaction sequence. The second sequence is represented in FIG. 2 and is termed as the green isolation sequence.

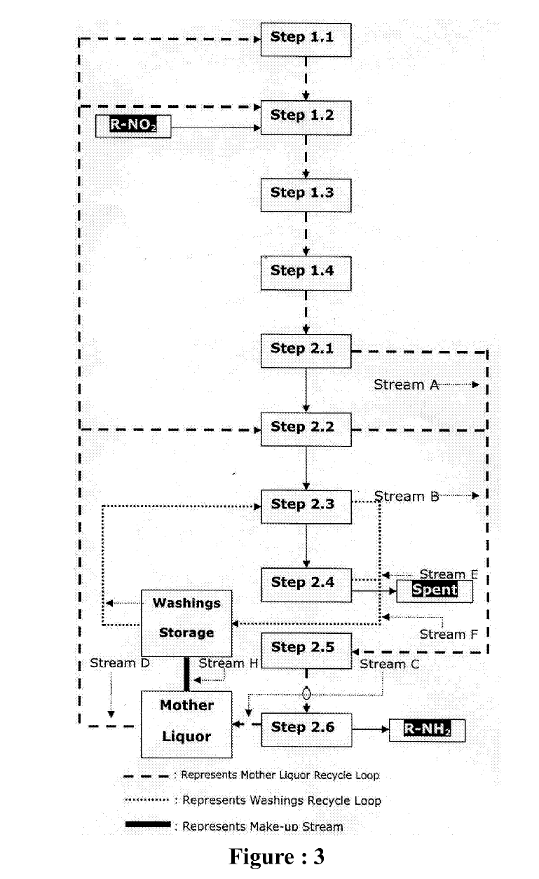

The combined process involved in first and second sequences as disclosed in the present invention is shown in FIG. 3. This figure shows the complete process of the present invention along with generation and fate of all liquid streams as disclosed in the present invention. One of the novel features of the process of present invention is regarding the reaction medium used in various stages of the process.

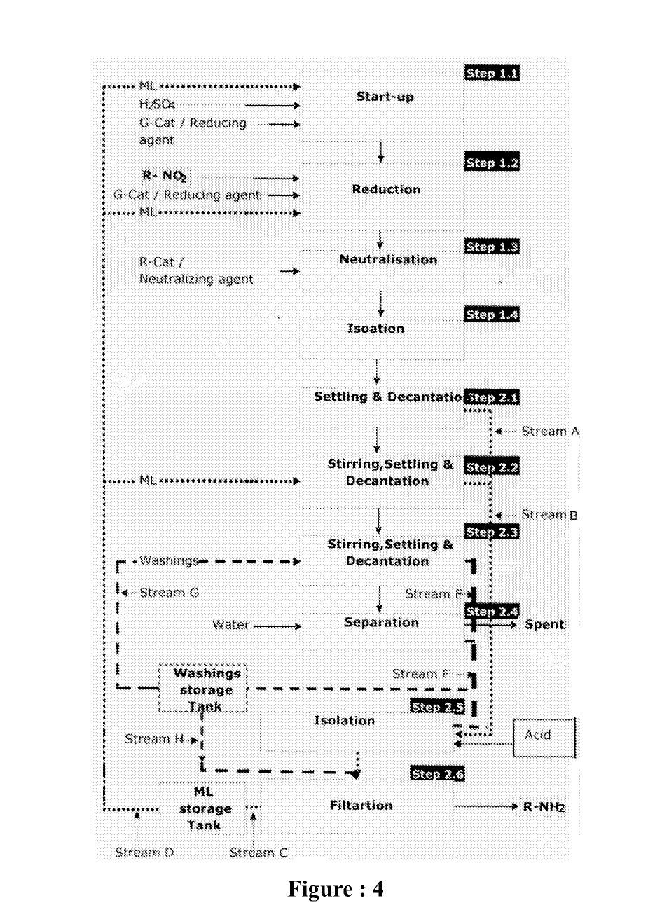

FIG. 4 shows the relationship between individual cycles of the process.

Referring to FIG. 3, In the very first cycle of the process, of the present invention FRM is used as the reaction medium in the start-up (Step 1.1) and reduction (Step 1.2) steps, and for steps involving washings (Steps 2.3, 2.4 & 2.6). As a key feature of the present invention, in the subsequent cycles, the liquid streams generated in various steps of recycling of the first cycle are used as the reaction medium. However, the use of these liquid streams as the reaction medium is optional and FRM can be used as the reaction medium in all cycles.

Streams generated at various stages of the invention are now defined. As shown in FIG. 3, Stream A is generated after settling and decantation steps that follow the reduction step. Stream B is generated after stirring, settling and decantation step. Both these streams (Stream A and B) are taken for isolation. Stream C is the mother liquor generated after the separation of R—NH₂ from the reaction medium. Stream C is stored in a mother liquor storage tank. Stream D is the liquid taken from mother liquor storage tank and which is used at various stages such as start-up, reduction and stirring, settling & decantation. Stream E is generated after stirring, settling & decantation. Stream F is generated after separation and washing of the inorganic by-product. Both these streams (Stream E and F) are taken to washings storage tank. Stream G from washings storage tank goes to stirring, settling & decantation steps. Stream H is generated from washings storage tank and goes to the mother liquor storage tank as make-up stream.

Some quantity of FRM or any other appropriate liquid streams, or a combination thereof are used as make-up liquid in various steps to compensate for the various liquid losses through handling, evaporation, and so on.

Details of the steps involved in the two sequences that form a typical cycle of the process of the present invention are described below, with reference to FIGS. 1, 2, 3, 4 and 5.

The preferred embodiment of the present invention and various other embodiments are now described.

Sequence 1.0—Green Reaction Sequence:

As shown in FIG. 1, this sequence comprises four steps, namely the start-up, reduction, neutralization, and isolation. Each of these steps is described below. One of the key features of this sequence is the various reaction agents that are used in various steps. These are the reducing agent and a neutralization agent. A predetermined quantity of these agents is added as appropriate. These agents along with the specific reaction conditions generated as defined by the temperature, pressure, pH, agitation, and other such parameters lead to the unique inherent recyclability of the process of the present invention.

The total quantity of the reducing agent required in this sequence for a typical cycle (referred to hereafter as Q_{RT}) is dictated by the requirement of the reduction potential of R—NO₂ or R—NO to be reduced. Q_{RT} is determined by a reducing agent's weight ratio, (Weight Ratio)_{RA}, that is the ratio of the weight of the reducing agent required in a single cycle, W_{RA} of the process of this invention to the weight of total amount of R—NO₂ or R—NO to be reduced in that single cycle, W_{N}. That is for a single cycle:

(Weight Ratio)_{RA}=W_{RA}/W_{N} Equation 1

The Q_{RT} is such that its weight is equal to W_{RA} which is determined from Equation 1.

In the preferred embodiment of the present invention, (Weight Ratio)_{RA} is preferably in the range of 0.25 to 25, the more preferable range being 0.5 to 2.5.

In the conventional processes, the power failures or other breakdowns of the plant lead to wastage of the entire batches leading to economical loss and environmental damage. One of the novel advantageous features of the process of this invention is that it allows recycling of the R—NH2 even without the stirring or agitation in many of its steps, particularly steps 1.2-1.4 and 2.2-2.5.

Step 1.1—Start-up: This step is carried out in a reaction vessel that has an agitator and necessary attachments known to a person skilled in the art. As shown in FIG. 1, at the start of the first cycle of process of the present invention an RM is charged to the reaction vessel in suitable quantities.

In the preferred embodiment of the present invention FRM is used as the reaction medium. The total quantity of the reaction medium required for a typical cycle (referred to hereafter Q_{RMT}) is dictated by the solubility of R—NH₂. This quantity is determined by weight ratio of FRM or the reaction medium, denoted as (Weight Ratio)_{RM}, that is the ratio of the weight of the FRM or the reaction medium required in a single cycle, W_{RM}, to the weight of total amount of R—NO₂ or R—NO to be reduced in that single cycle, W_{N}. That is

(Weight Ratio)_{RM}=W_{RM}/W_{N} Equation 2

The Q_{RMT} is such that its weight is equal to W_{RM} which is determined from Equation 2.

The quantity of the FRM or the reaction medium used in Step 1.1, denoted as Q_{RM1.1}, is variable.

In the preferred embodiment, (Weight Ratio)_{RM} is preferably in the range of 5 to 100, the more preferable range being 10 to 75, and Q_{RM1.1} is in the range of 0% (w/w) to 40% (w/w) of Q_{RMT} used in this cycle.

Optionally, a suitable acid, or a salt of iron with inorganic or organic acids, such as ferrous sulphate, ferrous chloride, ferrous ammonium sulphate, ferrous oxalate, ferrous citrate or other salts like ammonium chloride, ammonium sulphate, other such salts, or any combination of these, is added in suitable quantity and suitable form while maintaining temperature of the reaction mixture in the reaction vessel in the range of 0° C. to 200° C. In the preferred embodiment of the invention, sulfuric acid is used. In another embodiment of the present invention, the temperature at which the acid charged is in the range of 10° C. to 100° C., more preferably 50° C. to 100° C.

The mixture is agitated for a predetermined time that is in the range of 0 minutes to 5 hours, more preferably between 0.5 hours to 2.5 hours. In the agitation stage, the pH of the reaction mixture is maintained throughout at a predetermined level that is in the range of 1 to 9, preferable range being 2 to 7

At the end of the agitation stage, a reducing agent, preferably G-CAT is charged in suitable quantity. It is added either in its full required quantity or in any number of batches of any size, or continuously, or any combination thereof. The reducing agent is added over a predetermined period, at a predetermined temperature, and a predetermined pH. The period over which the reducing agent is added in the range of 0 minutes to 5 hours, more preferably between 0.5 hours to 2.5 hours. The temperature at which the reducing agent is added is in the range of 0° C. to 200° C. The pH at which the reducing agent is added is in the range of 1 to 9, preferable range being 2 to 7

In the preferred embodiment of the invention, G-CAT is used as the reducing agent and (Weight Ratio)_{RA} is preferably in the range of 0.25 to 25, the more preferable range being 0.5 to 2.5. The quantity of the reducing agent used in Step 1.1, Q_{R1.1} is variable in the range of 0% (w/w) to 100% (w/w) Q_{RT}.

In the subsequent cycles of the process of the present invention, Stream D is used as a reaction medium instead of FRM in the start-up (step 1.1).

In another embodiment of the present invention, the temperature at which the reducing agent is charged is in the range of 10° C. to 100° C., more preferably 50° C. to 100° C. G-CAT or any other customized proprietary catalytic formulation is used as the reducing agent in this embodiment.

In another embodiment of the present invention, the reaction medium, the acid, and the reducing agent are added in any sequence.

Step 1.2—Reduction: The nitro or nitroso compound (respectively R—NO₂ or R—NO) to be reduced is added to the reaction vessel either in its full quantity or in any number of lots. The total amount of R—NO₂ or R—NO to be reduced is added over a period of 0 to 25 hours, at a suitable interval that depends on the molecule to be reduced.

A reaction medium is charged in suitable quantity to the reaction vessel while maintaining the temperature, and pH of the mixture in their respective predetermined ranges. The temperature at which the reaction medium is added is in the range of 0° C. to 200° C. The pH at which the reaction medium is added is in the range of 1 to 9, preferable range being 2 to 7. In the preferred embodiment of the present invention, FRM is used as the reaction medium in step 1.2 of the first cycle of the process of the present invention. For subsequent cycle, Stream D is used as the reaction medium for this step.

The quantity of the FRM or the reaction medium used in Step 1.2, denoted as Q_{RM1.2}, is variable in the range of 0% (w/w) to 40% (w/w) of the total quantity Q_{RMT} used in this cycle.

A suitable acid is optionally added to the reaction vessel to achieve the desired pH level of the reaction mixture. The acid is added in a suitable form while maintaining temperature of the reaction mixture in the reaction vessel in the range of 0° C. to 200° C.

A reducing agent is charged to the reaction mixture. The reducing agent is charged over a period of time at a predetermined temperature which is in the range of 0° C. to 200° C., when the pH of the reaction mixture is at a predetermined value which is in the range of 1 to 9, preferable range being 2 to 7. The reducing agent required for Step 1.2 is added either in a single lot or in batches, or continuously, or any combination of these methods of addition.

The quantity of the reducing agent used in this step, denoted as Q_{RA1.2}, is variable in the range of 0% (w/w) to 100% (w/w) of the Q_{RT}. The quantity Q_{R1.2} is dictated by the requirement of the reduction potential for the R—NO₂ or R—NO to be reduced. The quantity Q_{RA1.2} is further determined so that it is the difference between Q_{RAT} and Q_{RA1.1}. That is:

Q_{RA1.2}=Q_{RAT}−Q_{RA1.1} Equation 3

In the preferred embodiment of the invention, G-CAT is used as the reducing agent and (Weight Ratio)_{RA} is preferably in the range of 0.25 to 25, the more preferable range being 0.5 to 2.5. In another embodiment of the present invention, any other reducing agent such as any proprietary agents is used as the reducing agent.

The reaction mixture at all stages of this step is optionally stirred with agitators rotating at a rate between 0 to 500 RPM. The step can be successfully carried out even without stirring.

Step 1.3—Neutralisation: After completion of the reduction at the end of Step 1.2, optionally a suitable reaction medium is charged in suitable quantity to the reaction vessel. The decision to add the reaction medium depends on the consistency of the solids. The quantity of the reaction medium used in Step 1.3, denoted as Q_{RM1.3}, is variable in the range of 0% (w/w) to 40% (w/w) of the total quantity Q_{RMT} used in this cycle.

A neutralizing agent is added to the reaction mixture over a predetermined period and at a predetermined temperature to adjust its pH to a suitable level. The fundamental role of the neutralisation agent is to provide a strong reduction potential for low concentration R—NO₂ or R—NO tailing towards the end step 1.2 and providing the necessary neutralisation for the reaction mixture obtained at the end of step 1.2.

The period over which the neutralising agent is added is in the range of 0 minutes to 5 hours, more preferably between 0.5 hours to 2.5 hours. The temperature at which the reducing agent is added is in the range of 0° C. to 200° C. The pH at which the reducing agent is added is in the range of 1 to 12, preferably 4 to 11.

The neutralisation process wherein the neutralisation agent is allowed to react with R—NO₂ or R—NO at a neutralisation process temperature and pH is continued for a neutralisation process time. The neutralisation process temperature is maintained in the range of 0° C. to 200° C., and the neutralisation process pH is maintained between 1 to 12, preferably 4 to 11. The neutralisation process time is in the range of 0 hours to 10 hours, preferably in the range of 30 minutes to 5 hours.

In the preferred embodiment of the process of the invention the neutralising agent is in the form of a formulation that comprises R-Cat or G-Cat or any combination thereof branded or unbranded. The quantity of the neutralising agent, Q_{NAT} is determined by its weight ratio, denoted as (Weight Ratio)_{NA}, that is the ratio of the weight of the neutralising agent required in a single cycle, W_{NA}, to the weight of total amount of R—NO₂ or R—NO to be reduced in that single cycle. That is,

(Weight Ratio)_{NA}=W_{NA}/W_{N} Equation 4

The Q_{NAT} is such that its weight is equal to W_{NA} which is determined from Equation 3.

(Weigh Ratio)_{NA} is preferably in the range of 0 to 2.5, more preferably between 0.05 to 0.25.

In the preferred embodiment of the invention, FRM is used as the reaction medium in the first cycle of this sequence, and for the subsequent cycles subject to the process of this invention, FRM is replaced by stream D.

In another embodiment of the present invention, any other neutralisation agents, proprietary or generic branded or unbranded, are used. Neutralizing agent in the form of hydroxides of alkali metals like sodium hydroxide, potassium hydroxide, calcium hydroxide, lithium hydroxide, carbonates or bicarbonates of alkali metals like sodium carbonate, sodium bicarbonate, potassium carbonate, potassium bicarbonate, calcium carbonate, lithium carbonate, other such salts or any combination thereof is optionally used as a neutralising agent.

The inventors have surprisingly found that the action of R-Cat and G-Cat in the steps 1.1 to 1.3 collectively favours very high degree of chemo-selectivity and regio-selectivity for R—NO₂ or R—NO to R—NH₂ green reduction reaction.

The reaction mixture at all stages of this step is optionally stirred with agitators rotating at a rate between 0 to 500 RPM. The step can be successfully carried out even without stirring.

Step 1.4—Isolation: R-Cat and or G-Cat is added to the reaction mixture. The mixture thus formed is termed as the Isolation mixture. The quantity of the R-Cat and or G-Cat is in the range of 0% (w/w) to 5% (w/w) of R—NO₂ or R—NO, preferable range being 0.5% (w/w) to 2.5% (w/w). It is charged at a predetermined Isolation temperature and at predetermined Isolation pH.

In the preferred embodiment of the present invention, the isolation temperature is in the range of 0° C. to 200° C., and the isolation pH is in the range of 3 to 14, preferably 4 to 12 more preferably 7 to 11

The pH and temperature conditions are maintained at this level of pH and temperature for predetermined time that is in the range of 0 hours to 24 hours, preferably in the range of 30 minutes to 5 hours.

Optionally a reaction medium is added to the Isolation mixture after or along with the addition of the R-Cat and or G-Cat. It is added at a predetermined temperature which is in the range of 0° C. to 200° C. and pH that is in the range of 1 to 14, preferably 4 to 12 more preferably 7 to 11

For the subsequent cycles subject to the process of this invention, FRM is replaced by stream D.

In all of the above steps, that is steps 1.1 to 1.4, sulfuric acid or any other mineral acid in dilute to concentrated form is used as the preferred suitable acid.

In another embodiment of the present invention, FRM is used as the reaction medium in the first cycle of the sequence. The quantity of the FRM or the reaction medium used in Step 1.4 Q_{RM1.4} is variable in the range of 0% (w/w) to 40% (w/w) of QRMT used in this cycle.

The reaction mixture at all stages of this step is optionally stirred with agitators rotating at a rate between 0 to 500 RPM. The step can be successfully carried out even without stirring.

A single cycle of the green reaction sequence is complete at the end of step 1.4. In another embodiment of the present invention, the purification temperature is preferably between 0° C. to 100° C.

Sequence 2.0—Green Isolation Sequence:

As shown in FIG. 2, this sequence comprises six steps, namely, a settling and decantation step, followed by two steps of stirring/settling/decantation, a separation & washings step, followed by an isolation step and a separation step. Each of these steps is described in detail.

Step 2.1—Settling and Decantation: As shown in FIG. 2, a reaction medium, referred to as the first settling RM, is optionally charged to the mixture obtained at the end of Step 1.4 in the reaction vessel in a suitable quantity and at suitable temperature and pH, the temperature being in the range of 0 to 200 and the pH being in the range of 1 to 12, preferably 4 to 11. The mixture thus formed is allowed to settle at a first settling pH, by maintaining it at a first settling temperature for a first settling time.

In the preferred embodiment of the invention the first settling pH is in the range of 1 to 12, preferably 4 to 11; the first settling temperature is in the range of 0° C. and 200° C., preferably between 0° C. to 100° C.; and the first settling time is for 1 minute to 10 hours, preferably 30 minutes to 3 hours.

Liquid layer that forms as a result of the settling process is decanted at a first decanting temperature, first decanting pH and first decanting time and charged as Stream A to Step 2.5 of same cycle or any of the following cycles.

In the preferred embodiment of the invention, the first decanting temperature is in the range between 0 to 200, more preferably between 0 to 100, first decanting pH between 1 to 12, preferably between 4 to 11.

In the preferred embodiment of the invention FRM is used as the reaction medium in the first cycle, and for the subsequent cycles subject to the process of this invention, FRM is replaced by stream D.

The quantity of the FRM or the reaction medium used in Step 2.1, denoted as Q_{RM2.1} is variable in the range of 0% (w/w) to 60% (w/w) of the Q_{RMT} used in this cycle.

Step 2.2—Stirring, Settling, and Decantation: The settled mass at the end of step 2.1 of the first cycle is charged with a predetermined quantity of a reaction medium, referred to as a second settling RM, at a predetermined first stirring temperature and a predetermined first stirring pH at a predetermined first stirring time. The mixture is stirred. Stirring is continued by maintaining the mixture at a predetermined first stirring continuation temperature, a predetermined first stirring continuation pH for a predetermined first stirring continuation time. Stirring is stopped and mass is allowed to settle at a predetermined second settling pH, a predetermined second settling temperature for a predetermined second settling time.

Once the solids are settled, the liquid layer collected at the top (referred to as Stream B) is decanted at a predetermined second decantation temperature, a predetermined second decantation pH and at a predetermined second decantation time and charged to Step 2.5 of the same cycle or any of the following cycles.

In the preferred embodiment of the invention, FRM is used in the first cycle as the second settling RM, and for the subsequent cycles subject to the process of this invention, FRM is replaced by stream D; the values of the first stirring temperature, the first stirring continuation temperature, and the second decantation temperature are in the range of 0° C. and 200° C., preferably between 0° C. to 100° C.; the values of the first stirring pH, the first stirring continuation pH, and the second decantation pH are in the range of 1 to 12, preferably between 4 to 11; the values of the first stirring time, the first stirring continuation time, and the second decantation time are in the range of 5 minutes to 5 hours; preferably 30 minutes to 3 hours.

The quantity of the FRM or the reaction medium used in Step 2.2, denoted as Q_{RM2.2} is variable in the range of 0% (w/w) to 60% (w/w) of Q_{RMT} used in this cycle.

Step 2.3—Stirring, Settling, and Decantation:

The settled mass at the end of step 2.2 of the first cycle is charged with a predetermined quantity of the reaction medium, referred to as a third settling RM, at a predetermined second stirring temperature and a predetermined second stirring pH at a predetermined second stirring time. The mixture is stirred. Stirring is continued by maintaining the mixture at a predetermined second stirring continuation temperature, a predetermined second stirring continuation pH for a predetermined second stirring continuation time. Stirring is stopped and mass is allowed to settle at a predetermined third settling pH, a predetermined third settling temperature for a predetermined third settling time.

Once the solids are settled, the liquid layer collected at the top (referred to as Stream E) is decanted at a predetermined third decantation temperature, a predetermined third decantation pH and at a predetermined third decantation time and charged to a washings storage tank.

In the preferred embodiment of the invention, FRM is used in the first cycle as the third settling RM, and for the subsequent cycles subject to the process of this invention, FRM is replaced by stream G; the values of the second stirring temperature, the second stirring continuation temperature, and the third decantation temperature are in the range of 0° C. and 200° C., preferably between 0° C. to 100° C.; the values of the second stirring pH, the second stirring continuation pH, and the third decantation pH are in the range of 1 to 12, preferably between 4 to 11; the values of the second stirring time, the second stirring continuation time, and the third decantation time are in the range of 5 minutes to 5 hours; preferably 30 minutes to 3 hours.

The quantity of the FRM or the reaction medium used in Step 2.3, denoted as Q_{RM2.3} is variable in the range of 0% (w/w) to 60% (w/w) of Q_{RMT} used in this cycle.

As a novel feature of the present invention, Step 2.3, which is similar to step 2.2, is carried out to ensure maximum removal of R—NH₂ by the reaction medium from the inorganic by-product.

Step 2.4—Separation and Washing: In all cycles of the isolation sequence, FRM in suitable quantity is charged into the reaction vessel to the solids obtained at the end of step 2.3. The separation mixture thus obtained is stirred. Stirring is continued at a predetermined separation temperature, a predetermined separation pH for a predetermined separation time. Stirring is stopped and solid mass is separated by known methods of solid liquid separation. Liquid stream obtained at the end of step 2.4 charged as Stream F to the washings storage tank.

In the preferred embodiment, the separation temperature sis in the range between 0° C. and 200° C., preferably between 0° C. to 100° C., the pH is between 1 to 12, preferably between 4 to 9, and the separation time is between5 minutes to 5 hours; preferably 30 minutes to 3 hours.

The quantity of the FRM used in Step 2.4 Q_{RM2.4} is variable in the range of 0% (w/w) to 60% (w/w) of the Q_{RMT} used in this cycle.

Step 2.5—R—NH₂ Isolation: Combined liquid streams (Stream A from Step 2.1 and Stream B from Step 2.2) obtained during this sequence are charged into another reaction vessel with agitator and other attachments known to a person skilled in the art.

In the preferred embodiment, R—NH₂ is separated from the reaction medium by predetermined isolation temperature and predetermined pH in the range of 4.0 to 12.0, preferable 7.0 to 11 by adding alkali of the concentration 1% (w/w) to 100% (w/w), preferably 1% (w/w) to 70% (w/w) more preferably 10% (w/w) to 60% (w/w) in the predetermined time in the range of 5 minutes to 10 hrs, preferably 30 minutes to 5 hours. Product is then separated from the reaction mass by adjusting pH in range of 1 to 8, preferably 2.0 to 7.0 using acid such as Sulphuric acid, Hydrochloric acid, phosphoric acid & other mineral acids branded or unbranded and combinations thereof.

Step 2.6—Isolation: Total mass obtained from Step 2.5 at predetermined isolation temperature and predetermined isolation pH is then separated by methods known to the person skilled in the art and washed with suitable quantity of FRM. The liquid and washings together (stream C) is stored in a mother liquor storage tank that contains liquid from any of earlier cycles.

In the preferred embodiment the isolation temperature is between 0° C. and 200° C., preferably between 50° C. to 100° C., and the pH between 3 to 14, preferably between 4 to 12.

A key advantageous feature of the present invention is that a part of the stored liquid, said part being defined as the stream D, in suitable quantity is recycled into various steps (Step 1.1 to 1.4 & 2.2) of the following cycles of the process of the invention.

The quantity of the FRM used in Step 2.6, denoted as Q_{RM2.6} is variable in the range of 0% (w/w) to 60% (w/w) of Q_{RMT} used in this cycle.

A typical cycle of the process of the present invention, that is a cycle consisting a green reaction sequence and a green isolation sequence, ends here. A key feature of the present invention is that all steps of a typical cycle are carried out at atmospheric pressure.

As a key advantageous feature of the present invention, the reaction medium used both the green reaction sequence and the green isolation sequence, in the cycles after the first cycle is taken from the mother liquor and various streams generated during the process of this invention. In other words, the FRM used in the various stages (Steps 1.1 to 1.4 and 2.1 to 2.3) of the first cycle is replaced by a suitable reaction medium in all subsequent cycles.

In steps 2.4 and 2.6 FRM is used in all cycles to make up the losses of previous cycles in the process of this invention.

The R—NH₂ is dried by any method selected from known methods of drying at predetermined temperature for predetermined time. This marks the completion of a single cycle of the process of the invention.

The inventors of the present invention have found that the purity of R—NH₂ after drying in any cycle varies in the range of 75% to 100%, preferably 80% to 99.9%.

The mother liquor and washings obtained during various steps described above are stored for processing in further cycles, number of recycles being generally in the range of 3 to 100 and above.

The inventors have surprisingly found that the reduction of R—NO₂ or R—NO to R—NH₂ carried out with the process described above generates inorganic by-product in any cycle in the ratio of weight in the range of 0.25 to 25 to the weight of R—NO₂ or R—NO to be reduced of the above sequence is crystalline and non-sticky in nature. Colour of the by-product ranges from light brown to jet-black particularly jet-black. The pH of the inorganic by-product is in the range of 4.0 to 8.0. The moisture content of the inorganic byproduct is in the range of 5% to 50%, particular range being 10% to 30%.

The inventors have found that the process of the present invention is applicable to the R—NO₂ or R—NO compounds having one sulphonic or carboxylic group and one or more nitro groups including aromatic R—NO₂ or R—NO compounds like nitrobenzene, nitronaphthalenes, nitroanthracenes, nitrophenanthrenes, heterocyclic nitro compounds with one or more hetero atoms either same or different, aliphatic nitro compounds and all such other nitro compounds.

The following table (Table 01) illustrates the savings in the various quantities of fresh water used and mother liquor recycled in the reaction of the present invention FC Acid.

| | | H2O & ML | | | NRC & NPC | | | | |
|---|---|---|---|---|---|---|---|---|---|
| | | consumption & saving | | | consumption | | | | |
| | Basis | | Treated | | | Soda | Reduction | Dry | |
| | [1] | | mother liquor | | NRC | solution | Temp | Product | Yield |
| Cycle | gm | H2O | ML | From | gm | gm | ° c. | (gm) | % |
| 0 | 50 | 850 | 0 | \acute{ } | 37.5 | 17 | 98 | 35 | 70 |
| 1 | 50 | 0 | 850 | Cycle 0 | 37.5 | 23 | 98 | 38 | 76 |
| 2 | 50 | 0 | 850 | Cycle 1 | 37.5 | 25 | 98 | 41 | 82 |
| 3 | 50 | 0 | 850 | Cycle 2 | 37.5 | 29 | 98 | 39 | 78 |
| 4 | 50 | 0 | 850 | Cycle 3 | 37.5 | 25 | 98 | 40.5 | 81 |
| 5 | 50 | 0 | 850 | Cycle 4 | 37.5 | 32 | 98 | 40 | 80 |

### EXAMPLE 1

### 4 Nitro 4-amine diphenyl amine 2 sulphonic acid to 4,4 diamine diphenylamine 2 sulphonic acid

Fresh cycle: In a round bottom flask equipped with stirrer, condenser, thermometer, addition port arranged in suitable heating/cooling system was charged 400 ml water, heated to 98° C. Charged 2 ml 98% H2SO4 to get pH 2.0 and 9.5 g G-CAT start up with continuous stirring at 98° C. First lot of 5.60 g G-CAT and first lot of 14.2 g Nitro was charged to the reaction mass in 10 min. The reaction mass was maintained for 5-10 min at 98° C. Remaining G-CAT & Nitro was charged in four equal lots in similar manner as followed for first lot. Reaction mass was maintained at 98° C. for 30 min. 17.0 g Na2CO3 was charged during 60 min to adjust pH of reaction mass to 10.5 and maintain for 15-20 minutes. Then it is decanted and collected in separate flask for further processing. Charged 300 ml water was charged for extraction, maintained temperature 98° C. and stirring was stopped and upper liquid layer was decanted to the crystallizer. Again 150 ml water was added to solid inorganic by-product, remaining in the flask under stirring at 98° C., to get 62.0 g of wet cake of solid inorganic by-product with moisture 22.6% & 0.96% amine content, appearance was black. The decanted mass is the cooled to 30-35° C. and charged 37 ml 33% Sulphuric acid (H2SO4) slowly in 2-3 hrs and then maintained for 2 hrs at pH 5.5. Liquid layer in the crystallizer was cooled to room temperature, & maintained for 2 hours; crystalline material was filtered, to get on drying 35.00 g of bluish-Violet powder colour with purity 94.33%. Total filtrate i.e. reaction medium and washings was collected & recycled in subsequent batches.

First Recycle (R1): In the same set up as described above, 400 ml reaction medium generated in fresh cycle was charged, heated to 98° C. Charged 2 ml 98% H2SO4 to get pH 2.0 and 9.5 g G-CAT start up with continuous stirring at 98° C. First lot of 5.60 g G-CAT and first lot of 14.2 g Nitro was charged to the reaction mass in 10 min. The reaction mass was maintained for 5-10 min at 98° C. Remaining G-CAT & Nitro was charged in four equal lots in similar manner as followed for first lot. Reaction mass was maintained at 98° C. for 30 min 23.0 g Na2CO3 was charged during 60 min to adjust pH of reaction mass to 10.5 and maintain for 15-20 minutes. Then it is decanted and collected in separate flask for further processing. Charged 300 ml water was charged for extraction, maintained temperature 98° C. and stirring was stopped and upper liquid layer was decanted to the crystallizer. Again 150 ml water was added to solid inorganic by-product, remaining in the flask under stirring at 98° C., to get 68.0 g of wet cake of solid inorganic by-product with moisture 18% & 1.10% amine content, appearance was black. The decanted mass is the cooled to 30-35° C. and charged 52 ml 33% Sulphuric acid (H2SO4) slowly in 2-3 hrs and then maintained for 2 hrs at pH 5.5. Liquid layer in the crystallizer was cooled to room temperature, & maintained for 2 hours; crystalline material was filtered, to get on drying 38.00 g of bluish-violet powder with purity 90.49%. Total filtrate i.e. reaction medium and washings was collected & recycled in subsequent batches.

Second Recycle (R2): In the same set up as described above, 400 ml reaction medium generated in first cycle was charged, heated to 98° C. Charged 2 ml 98% H2SO4 to get pH 2.0 and 9.5 g G-CAT start up with continuous stirring at 98° C. First lot of 5.60 g G-CAT and first lot of 14.2 g Nitro was charged to the reaction mass in 10 min. The reaction mass was maintained for 5-10 min at 98° C. Remaining G-CAT & Nitro was charged in four equal lots in similar manner as followed for first lot. Reaction mass was maintained at 98° C. for 30 min 25.0 g Na2CO3 was charged during 60 min to adjust pH of reaction mass to 10.5 and maintain for 15-20 minutes. Then it is decanted and collected in separate flask for further processing. Charged 300 ml water was charged for extraction, maintained temperature 98° C. and stirring was stopped and upper liquid layer was decanted to the crystallizer. Again 150 ml water was added to solid inorganic by-product, remaining in the flask under stirring at 98° C., to get 68.0 g of wet cake of solid inorganic by-product with moisture 20% & 0.82% amine content, appearance was black. The decanted mass is the cooled to 30-35° C. and charged 58 ml 33% Sulphuric acid (H2SO4) slowly in 2-3 hrs and then maintained for 2 hrs at pH 5.5. Liquid layer in the crystallizer was cooled to room temperature, & maintained for 2 hours; crystalline material was filtered, to get on drying 41.00 g of bluish Violet-powder colour with purity 89.67%. Total filtrate i.e. reaction medium and washings was collected & recycled in subsequent batches.

Third Recycle (R3): In the same set up as described above, 400 ml reaction medium generated in second cycle was charged, heated to 98° C. Charged 2 ml 98% H2SO4 to get pH 2.0 and 9.5 g G-CAT start up with continuous stirring at 98° C. First lot of 5.60 g G-CAT and first lot of 14.2 g Nitro was charged to the reaction mass in 10 min. The reaction mass was maintained for 5-10 min at 98° C. Remaining G-CAT & Nitro was charged in four equal lots in similar manner as followed for first lot. Reaction mass was maintained at 98° C. for 30 min 29.0 g Na2CO3 was charged during 60 min to adjust pH of reaction mass to 10.5 and maintain for 15-20 minutes. Then it is decanted and collected in separate flask for further processing. Charged 300 ml water was charged for extraction, maintained temperature 98° C. and stirring was stopped and upper liquid layer was decanted to the crystallizer. Again 150 ml water was added to solid inorganic by-product, remaining in the flask under stirring at 98° C., to get 75.0 g of wet cake of solid inorganic by-product with moisture 26.60% & 0.83% amine content, appearance was black. The decanted mass is the cooled to 30-35° C. and charged 66 ml 33% Sulphuric acid (H2SO4) slowly in 2-3 hrs and then maintained for 2 hrs at pH 5.5. Liquid layer in the crystallizer was cooled to room temperature, & maintained for 2 hours; crystalline material was filtered, to get on drying 39.00 g of bluish-Violet-powder colour with purity 88.30%. Total filtrate i.e. reaction medium and washings was collected & recycled in subsequent batches.

Fourth Recycle (R4): In the same set up as described above, 400 ml reaction medium generated in third cycle was charged, heated to 98° C. Charged 2 ml 98% H2SO4 to get pH 2.0 and 9.5 g G-CAT start up with continuous stirring at 98° C. First lot of 5.60 g G-CAT and first lot of 14.2 g Nitro was charged to the reaction mass in 10 min. The reaction mass was maintained for 5-10 min at 98° C. Remaining G-CAT & Nitro was charged in four equal lots in similar manner as followed for first lot. Reaction mass was maintained at 98° C. for 30 min 25.0 g Na2CO3 was charged during 60 min to adjust pH of reaction mass to 10.5 and maintain for 15-20 minutes. Then it is decanted and collected in separate flask for further processing. Charged 300 ml water was charged for extraction, maintained temperature 98° C. and stirring was stopped and upper liquid layer was decanted to the crystallizer. Again 150 ml water was added to solid inorganic by-product, remaining in the flask under stirring at 98° C., to get 79.0 g of wet cake of solid inorganic by-product with moisture 15.18% & 0.78% amine content, appearance was black. The decanted mass is the cooled to 30-35° C. and charged 56 ml 33% Sulphuric acid (H2SO4) slowly in 2-3 hrs and then maintained for 2 hrs at pH 5.5. Liquid layer in the crystallizer was cooled to room temperature, & maintained for 2 hours; crystalline material was filtered, to get on drying 40.5 g of bluish-Violet powder colour with purity 87.20%. Total filtrate i.e. reaction medium and washings was collected & recycled in subsequent batches.

Fifth Recycle (R5): In the same set up as described above, 400 ml reaction medium generated in third cycle was charged, heated to 98° C. Charged 2 ml 98% H2SO4 to get pH 2.0 and 9.5 g G-CAT start up with continuous stirring at 98° C. First lot of 5.60 g G-CAT and first lot of 14.2 g Nitro was charged to the reaction mass in 10 min. The reaction mass was maintained for 5-10 min at 98° C. Remaining G-CAT & Nitro was charged in four equal lots in similar manner as followed for first lot. Reaction mass was maintained at 98° C. for 30 min 32.0 g Na2CO3 was charged during 60 min to adjust pH of reaction mass to 10.5 and maintain for 15-20 minutes. Then it is decanted and collected in separate flask for further processing. Charged 300 ml water was charged for extraction, maintained temperature 98° C. and stirring was stopped and upper liquid layer was decanted to the crystallizer. Again 150 ml water was added to solid inorganic by-product, remaining in the flask under stirring at 98° C., to get 68.0 g of wet cake of solid inorganic by-product with moisture 27.94% & 1.38% amine content, appearance was black. The decanted mass is the cooled to 30-35° C. and charged 69 ml 33% Sulphuric acid (H2SO4) slowly in 2-3 hrs and then maintained for 2 hrs at pH 5.5. Liquid layer in the crystallizer was cooled to room temperature, & maintained for 2 hours; crystalline material was filtered, to get on drying 40.0 g of bluish-Violet powder colour with purity 85.01%. Total filtrate i.e. reaction medium and washings was collected & recycled in subsequent batches.

The following table (Table 02) illustrates the savings in the various quantities of fresh water used and mother liquor recycled in the reaction of the present invention of MPDSA.

| | | H2O & ML | | | | | | | |
|---|---|---|---|---|---|---|---|---|---|
| | | consumption & saving | | | NRC & NGC | | | | |
| | Basis | | Treated | | consumption | | Reduction | Dry | |
| | [1] | | mother liquor | | NRC | NGC | Temp | Product | Yield |
| Cycle | g | H2O | ML | From | gm | gm | ° c. | (g) | % |
| 0 | 50 | 275 | 0 | \acute{ }0 | 70 | 4.0 | 95-100 | 27.50 | 55 |
| 1 | 50 | 0 | 375 | Cycle 0 | 70 | 5.0 | 95-100 | 29.50 | 59 |
| 2 | 50 | 0 | 340 | Cycle 1 | 70 | 5.0 | 95-100 | 29.00 | 58 |
| 3 | 50 | 0 | 400 | Cycle 2 | 70 | 5.0 | 95-100 | 29.50 | 59 |
| 4 | 50 | 0 | 445 | Cycle 3 | 70 | 5.0 | 95-100 | 30.00 | 60 |

### EXAMPLE 2

### Meta dinitro sulphonic acid to m-phenylenediamine 4-sulphonic acid

Fresh cycle: In a round bottom flask equipped with stirrer, condenser, thermometer, addition port arranged in suitable heating/cooling system was charged 175 ml water, heated to 80° C. Charged 10 ml (30%) HCl to get pH 2.0 and 25.0 g G-CAT start up with continuous stirring at 95-100° C. First lot of 9.0 g G-CAT and first lot of 12.8 g Nitro was charged to the reaction mass in 10 min. The reaction mass was maintained for 5-10 min at 95-100° C. Remaining G-CAT & Nitro was charged in four equal lots in similar manner as followed for first lot. Reaction mass was maintained at 95-100° C. for 30 min. Then charged 4.0 g R-Cat was charged during 30 min to adjust pH of reaction mass to 7.0 and maintain for 15-20 minutes. Then it is filtered and spent catalyst is again washed 100 ml hot water to get 129 g wet inorganic by-product with moisture 23% and amine content 0.70%. The combined mass of product & wash filtrate is the cooled to 35-40° C. and charged 23.5 ml 20% Sulphuric acid slowly to get pH 2.0. After adjusting pH the slurry is chilled to 20° C. and then filtered. The wet cake of product is washed with 75 ml Cold water to get on drying 27.50 g of Grey colour with purity 99.00% with melting point 265° C. Total filtrate i.e. reaction medium and washings was collected & recycled in subsequent batches after treatment.

First Recycle: In the same set up as described above, 275 ml reaction medium generated in fresh cycle was charged, heated to 80° C. Charged 11 ml (30%) HCl to get pH 2.0 and 25.0 g G-CAT start up with continuous stirring at 95-100° C. First lot of 9.0 g G-CAT and first lot of 12.8 g Nitro was charged to the reaction mass in 10 min. The reaction mass was maintained for 5-10 min at 95-100° C. Remaining G-CAT & Nitro was charged in four equal lots in similar manner as followed for first lot. Reaction mass was maintained at 95-100° C. for 30 min Then charged 5.0 g R-Cat was charged during 30 min to adjust pH of reaction mass to 7.0 and maintain for 15-20 minutes. Then it is filtered and spent catalyst is again washed 100 ml hot water to get 153 g wet inorganic by-product with moisture 31.37% and amine content 0.74%. The combined mass of product & wash filtrate is the cooled to 35-40° C. and charged 25.0 ml 20% Sulphuric acid slowly to get pH 2.0. After adjusting pH the slurry is chilled to 20° C. and then filtered. The wet cake of product is washed with 75 ml Cold water to get on drying 29.50 g of Grey colour with purity 99.00% with melting point 262° C. Total filtrate i.e. reaction medium and washings was collected & recycled in subsequent batches after treatment.

Second Recycle: In the same set up as described above, 240 ml reaction medium generated in first cycle was charged, heated to 80° C. Charged 10 ml (30%) HCl to get pH 2.0 and 25.0 g G-CAT start up with continuous stirring at 95-100° C. First lot of 9.0 g G-CAT and first lot of 12.8 g Nitro was charged to the reaction mass in 10 min. The reaction mass was maintained for 5-10 min at 95-100° C. Remaining G-CAT & Nitro was charged in four equal lots in similar manner as followed for first lot. Reaction mass was maintained at 95-100° C. for 30 min Then charged 5.0 g R-Cat was charged during 30 min to adjust pH of reaction mass to 7.0 and maintain for 15-20 minutes. Then it is filtered and spent catalyst is again washed 100 ml hot water to get 143 g wet inorganic by-product with moisture 23.07% and amine content 0.83%. The combined mass of product & wash filtrate is the cooled to 35-40° C. and charged 28.0 ml 20% Sulphuric acid slowly to get pH 2.0. After adjusting pH the slurry is chilled to 20° C. and then filtered. The wet cake of product is washed with 75 ml Cold water to get on drying 29.00 g of Grey colour with purity 99.00% with melting point 262° C. Total filtrate i.e. reaction medium and washings was collected & recycled in subsequent batches after treatment.

Third Recycle: In the same set up as described above, 300 ml reaction medium generated in second cycle was charged, heated to 80° C. Charged 10 ml (30%) HCl to get pH 2.0 and 25.0 g G-CAT start up with continuous stirring at 95-100° C. First lot of 9.0 g G-CAT and first lot of 12.8 g Nitro was charged to the reaction mass in 10 min. The reaction mass was maintained for 5-10 min at 95-100° C. Remaining G-CAT & Nitro was charged in four equal lots in similar manner as followed for first lot. Reaction mass was maintained at 95-100° C. for 30 min. Then charged 5.0 g R-Cat was charged during 30 min to adjust pH of reaction mass to 7.0 and maintain for 15-20 minutes. Then it is filtered and spent catalyst is again washed 100 ml hot water to get 143 g wet inorganic by-product with moisture 21.67njii % and amine content 0.74%. The combined mass of product & wash filtrate is the cooled to 35-40° C. and charged 32.0 ml 20% Sulphuric acid slowly to get pH 2.0. After adjusting pH the slurry is chilled to 20° C. and then filtered. The wet cake of product is washed with 75 ml Cold water to get on drying 29.50 g of Grey colour with purity 99.00% with melting point 261° C. Total filtrate i.e. reaction medium and washings was collected & recycled in subsequent batches after treatment.

Fourth Recycle: In the same set up as described above, 345 ml reaction medium generated in third cycle was charged, heated to 80° C. Charged 10 ml (30%) HCl to get pH 2.0 and 25.0 g G-CAT start up with continuous stirring at 95-100° C. First lot of 9.0 g G-CAT and first lot of 12.8 g Nitro was charged to the reaction mass in 10 min. The reaction mass was maintained for 5-10 min at 95-100° C. Remaining G-CAT & Nitro was charged in four equal lots in similar manner as followed for first lot. Reaction mass was maintained at 95-100° C. for 30 min Then charged 5.0 g R-Cat was charged during 30 min to adjust pH of reaction mass to 7.0 and maintain for 15-20 minutes. Then it is filtered and spent catalyst is again washed 100 ml hot water to get 154 g wet inorganic by-product with moisture 25.32% and amine content 0.74%. The combined mass of product & wash filtrate is the cooled to 35-40° C. and charged 32.0 ml 20% Sulphuric acid slowly to get pH 2.0. After adjusting pH the slurry is chilled to 20° C. and then filtered. The wet cake of product is washed with 75 ml Cold water to get on drying 30.00 g of Grey colour with purity 99.00% with melting point 264° C. Total filtrate i.e. reaction medium and washings was collected & recycled in subsequent batches after treatment.

The following table (Table 03) illustrates the savings in the various quantities of fresh water used and mother liquor recycled in the reaction of the present invention of DASDA.

| | | H2O & ML | | | | | | | |
|---|---|---|---|---|---|---|---|---|---|
| | | consumption & saving | | | NRC & NPC | | | | |
| | Basis | | Treated | | consumption | | Reduction | Dry | |
| | [1] | | mother liquor | | NRC | NGC | Temp | Product | Yield |
| Cycle | g | H2O | ML | From | g | g | ° c. | (g) | % |
| 0 | 50 | 575 | 0 | 0 | 40 | 4.0 | 95-100 | 30 | 60 |
| 1 | 50 | 0 | 700 | Cycle 0 | 40 | 3.0 | 95-100 | 40 | 80 |
| 2 | 50 | 0 | 780 | Cycle 1 | 40 | 3.0 | 95-100 | 38 | 64 |
| 3 | 50 | 0 | 780 | Cycle 2 | 40 | 3.0 | 95-100 | 36 | 72 |
| 4 | 50 | 0 | 750 | Cycle 3 | 40 | 3.0 | 95-100 | 32 | 64 |

### EXAMPLE 3

### 4-4 Di Nitro Stilbene-2-2 Disulphonic Acid (DNSDA) to 4-4 Di Amino Stilbene 2, 2 Di Sulphonic Acid (DASDA)

Fresh cycle: In a 1-Liter-4-neck round bottom flask equipped with stirrer, condenser, thermometer, addition port arranged in suitable heating/cooling system was charged 300 ml water, heated to 80° C. Charged 6 ml 30% H2SO4 to get pH 2.0 and immediately charged 40 g G-CAT start up with continuous stirring at 80° C. First lot of 15.2 g 4-4 Di Nitro Stilbene-2-2 Disulphonic Acid (DNSDA) was charged to the reaction mass in 10 min. The reaction mass was maintained for 5-10 min at 98-100° C. Remaining lots of 4-4 Di Nitro Stilbene-2-2 Disulphonic Acid (DNSDA) was charged in four equal lots in similar manner as followed for first lot. Reaction mass was maintained at 98-100° C. for 30 min. Then 5.0 g R-Cat was charged to adjust pH 8.5. Then it is filtered & collected in separate flask for further processing. 200 ml hot water wash given to solid inorganic by-product, remaining in the flask under stirring at 98°-100° C., to get 61.0 g of wet cake of solid inorganic by-product, with moisture content 19.0% & 0.32% amine content, and appearance was black. The decanted mass is then heated to 85° C. and charged 31 ml 20% Sulphuric acid (H2SO4) slowly in 0.5-1.0 hrs and then maintained for 2 hrs at pH 2.0. Liquid layer in the crystallizer was cooled to room temperature, & maintained for 2 hours; crystalline material was filtered and washed with 75 ml water to get 30 g Pale yellow 4-4 Di Amino Stilbene 2, 2 Di Sulphonic Acid (DASDA) with purity 94%. Total filtrate i.e. reaction medium and washings was collected & recycled in subsequent batches.

First recycle: In the same set up as described above, 500 ml reaction medium generated in fresh cycle was charged, heated to 90° C. Charged 5 ml 20% H2SO4 to get pH 2.0 and immediately charged 40 g G-CAT start up with continuous stirring at 98-100° C. First lot of 15.2 g 4-4 Di Nitro Stilbene-2-2 Disulphonic Acid (DNSDA) was charged to the reaction mass in 10 min. The reaction mass was maintained for 5-10 min at 98-100° C. Remaining lots of 4-4 Di Nitro Stilbene-2-2 Disulphonic Acid (DNSDA) was charged in four equal lots in similar manner as followed for first lot. Reaction mass was maintained at 98-100° C. for 30 min. Then 3.0 g R-Cat was charged to adjust pH 8.5. Then it is filtered & collected in separate flask for further processing. 200 ml hot water wash given to solid inorganic by-product, remaining in the flask under stirring at 98°-100° C., to get 64.0 g of wet cake of solid inorganic by-product, with moisture content 20.0% & amine content 0.76% , and appearance was black. The decanted mass is then heated to 85° C. and charged 31 ml 20% Sulphuric acid (H2SO4) slowly in 0.5-1.0 hrs and then maintained for 2 hrs at pH 2.0. Liquid layer in the crystallizer was cooled to room temperature, & maintained for 2 hours; crystalline material was filtered and washed with 75 ml water to get 40 g Pale yellow 4-4 Di Amino Stilbene 2, 2 Di Sulphonic Acid (DASDA) with purity 94%. Total filtrate i.e. reaction medium and washings was collected & recycled in subsequent batches.

Second recycle: In the same set up as described above, 580 ml reaction medium generated in first recycle was charged, heated to 90° C. Charged 5 ml 20% H2SO4 to get pH 2.0 and immediately charged 40 g G-CAT start up with continuous stirring at 98-100° C. First lot of 15.2 g 4-4 Di Nitro Stilbene-2-2 Disulphonic Acid (DNSDA) was charged to the reaction mass in 10 min. The reaction mass was maintained for 5-10 min at 98-100° C. Remaining lots of 4-4 Di Nitro Stilbene-2-2 Disulphonic Acid (DNSDA) was charged in four equal lots in similar manner as followed for first lot. Reaction mass was maintained at 98-100° C. for 30 min. Then 3.0 g R-Cat was charged to adjust pH 8.5. Then it is filtered & collected in separate flask for further processing. 200 ml hot water wash given to solid inorganic by-product, remaining in the flask under stirring at 98°-100° C., to get 63.0 g of wet cake of solid inorganic by-product, with moisture content 18.0% & amine content 0.97% , and appearance was black. The decanted mass is then heated to 85° C. and charged 55 ml 20% Sulphuric acid (H2SO4) slowly in 0.5-1.0 hrs and then maintained for 2 hrs at pH 2.0. Liquid layer in the crystallizer was cooled to room temperature, & maintained for 2 hours; crystalline material was filtered and washed with 75 ml water to get 38 g Pale yellow 4-4 Di Amino Stilbene 2, 2 Di Sulphonic Acid (DASDA) with purity 93%. Total filtrate i.e. reaction medium and washings was collected & recycled in subsequent batches.

Third recycle: In the same set up as described above, 580 ml reaction medium generated in second recycle was charged, heated to 90° C. Charged 6 ml 20% H2SO4 to get pH 2.0 and immediately charged 40 g G-CAT start up with continuous stirring at 98-100° C. First lot of 15.2 g 4-4 Di Nitro Stilbene-2-2 Disulphonic Acid (DNSDA) was charged to the reaction mass in 10 min. The reaction mass was maintained for 5-10 min at 98-100° C. Remaining lots of 4-4 Di Nitro Stilbene-2-2 Disulphonic Acid (DNSDA) was charged in four equal lots in similar manner as followed for first lot. Reaction mass was maintained at 98-100° C. for 30 min. Then 3.0 g R-Cat was charged to adjust pH 8.5. Then it is filtered & collected in separate flask for further processing. 200 ml hot water wash given to solid inorganic by-product, remaining in the flask under stirring at 98°-100° C., to get 63.0 g of wet cake of solid inorganic by-product, with moisture content 18.0% & amine content 0.97% , and appearance was black. The decanted mass is then heated to 85° C. and charged 56 ml 20% Sulphuric acid (H2SO4) slowly in 0.5-1.0 hrs and then maintained for 2 hrs at pH 2.0. Liquid layer in the crystallizer was cooled to room temperature, & maintained for 2 hours; crystalline material was filtered and washed with 75 ml water to get 36 g Pale yellow 4-4 Di Amino Stilbene 2, 2 Di Sulphonic Acid (DASDA) with purity 93%. Total filtrate i.e. reaction medium and washings was collected & recycled in subsequent batches.

Fourth recycle: In the same set up as described above, 550 ml reaction medium generated in third recycle was charged, heated to 90° C. Charged 6 ml 20% H2SO4 to get pH 2.0 and immediately charged 40 g G-CAT start up with continuous stirring at 98-100° C. First lot of 15.2 g 4-4 Di Nitro Stilbene-2-2 Disulphonic Acid (DNSDA) was charged to the reaction mass in 10 min. The reaction mass was maintained for 5-10 min at 98-100° C. Remaining lots of 4-4 Di Nitro Stilbene-2-2 Disulphonic Acid (DNSDA) was charged in four equal lots in similar manner as followed for first lot. Reaction mass was maintained at 98-100° C. for 30 min. Then 3.0 g R-Cat was charged to adjust pH 8.5. Then it is filtered & collected in separate flask for further processing. 200 ml hot water wash given to solid inorganic by-product, remaining in the flask under stirring at 98°-100° C., to get 62.0 g of wet cake of solid inorganic by-product, with moisture content 18.0% & amine content 0.92% , and appearance was black. The decanted mass is then heated to 85° C. and charged 60 ml 20% Sulphuric acid (H2SO4) slowly in 0.5-1.0 hrs and then maintained for 2 hrs at pH 2.0. Liquid layer in the crystallizer was cooled to room temperature, & maintained for 2 hours; crystalline material was filtered and washed with 75 ml water to get 32 g Pale yellow 4-4 Di Amino Stilbene 2, 2 Di Sulphonic Acid (DASDA) with purity 93%. Total filtrate i.e. reaction medium and washings was collected & recycled in subsequent batches.

The following table (Table 04) illustrates the savings in the various quantities of fresh water used and mother liquor recycled in the reaction of the present invention (DABA).

| | | | | | NRC & Soda | | | | |
|---|---|---|---|---|---|---|---|---|---|
| | | H2O & ML | | | solution | | | | |
| | | consumption & saving | | | consumption | | | | |
| | Basis | | Treated | | | Soda | Reduction | Dry | |
| | [1] | | mother liquor | | NRC | solution | Temp | Product | Yield |
| Cycle | g | H2O | ML | From | g | 25% ml | ° c. | (g) | % |
| 0 | 100 | 1500 | 0 | 0\acute{ } | 200 | 160 | 98 | 42 | 42 |
| 1 | 100 | 0 | 1500 | Cycle 0 | 200 | 165 | 98 | 63 | 63 |
| 2 | 100 | 0 | 1500 | Cycle 1 | 200 | 170 | 98 | 64 | 64 |
| 3 | 100 | 0 | 1500 | Cycle 2 | 200 | 160 | 98 | 70 | 70 |
| 4 | 100 | 0 | 1500 | Cycle 3 | 200 | 170 | 98 | 66 | 66 |
| 5 | 100 | 0 | 1500 | Cycle 4 | 200 | 160 | 98 | 70 | 70 |
| 6 | 100 | 0 | 1500 | Cycle 5 | 200 | 175 | 98 | 68 | 68 |
| 7 | 100 | 0 | 1500 | Cycle 6 | 200 | 175 | 98 | 65 | 65 |
| 8 | 100 | 0 | 1500 | Cycle 7 | 200 | 180 | 98 | 71 | 71 |
| 9 | 100 | 0 | 1500 | Cycle 8 | 200 | 200 | 98 | 65 | 65 |
| 10 | 100 | 0 | 1500 | Cycle 9 | 200 | 180 | 98 | 67 | 67 |
| 11 | 100 | 0 | 1500 | Cycle 10 | 200 | 180 | 98 | 75 | 75 |
| 12 | 10 | 0 | 1500 | Cycle 11 | 200 | 190 | 98 | 66 | 66 |
| 13 | 100 | 0 | 1500 | Cycle 12 | 200 | 195 | 98 | 70 | 70 |
| 14 | 100 | 0 | 1500 | Cycle 13 | 200 | 200 | 98 | 62 | 62 |
| 15 | 100 | 0 | 1500 | Cycle 14 | 200 | 195 | 98 | 58 | 58 |
| 16 | 100 | 0 | 1500 | Cycle 15 | 200 | 200 | 98 | 65 | 65 |
| 17 | 10 | 0 | 1500 | Cycle 16 | 200 | 210 | 98 | 70 | 70 |
| 18 | 10 | 0 | 1500 | Cycle 17 | 200 | 200 | 98 | 65 | 65 |
| 19 | 100 | 0 | 1500 | Cycle 18 | 200 | 200 | 98 | 66 | 66 |
| 20 | 10 | 0 | 1500 | Cycle 19 | 200 | 200 | 98 | 65 | 65 |
| 21 | 10 | 0 | 1500 | Cycle 20 | 200 | 200 | 98 | 69 | 69 |
| 22 | 10 | 0 | 1500 | Cycle 21 | 200 | 200 | 98 | 65 | 65 |
| 23 | 10 | 0 | 1500 | Cycle 22 | 200 | 5195 | 98 | 68 | 68 |
| 24 | 100 | 0 | 1500 | Cycle 23 | 200 | 195 | 98 | 65 | 65 |
| 25 | 100 | 0 | 1500 | Cycle 24 | 200 | 195 | 98 | 66 | 66 |
| 0 | 100 | 1500 | 0 | 0\acute{ } | 200 | 160 | 98 | 42 | 42 |
| 1 | 100 | 0 | 1500 | Cycle 0 | 200 | 165 | 98 | 63 | 63 |
| 2 | 100 | 0 | 1500 | Cycle 1 | 200 | 170 | 98 | 64 | 64 |
| 3 | 100 | 0 | 1500 | Cycle 2 | 200 | 160 | 98 | 70 | 70 |
| 4 | 100 | 0 | 1500 | Cycle 3 | 200 | 170 | 98 | 66 | 66 |
| 5 | 100 | 0 | 1500 | Cycle 4 | 200 | 160 | 98 | 70 | 70 |
| 6 | 100 | 0 | 1500 | Cycle 5 | 200 | 175 | 98 | 68 | 68 |
| 7 | 100 | 0 | 1500 | Cycle 6 | 200 | 175 | 98 | 65 | 65 |
| 8 | 100 | 0 | 1500 | Cycle 7 | 200 | 180 | 98 | 71 | 71 |
| 9 | 100 | 0 | 1500 | Cycle 8 | 200 | 200 | 98 | 65 | 65 |
| 10 | 100 | 0 | 1500 | Cycle 9 | 200 | 180 | 98 | 67 | 67 |
| 11 | 100 | 0 | 1500 | Cycle 10 | 200 | 180 | 98 | 75 | 75 |
| 12 | 10 | 0 | 1500 | Cycle 11 | 200 | 190 | 98 | 66 | 66 |
| 13 | 100 | 0 | 1500 | Cycle 12 | 200 | 195 | 98 | 70 | 70 |
| 14 | 100 | 0 | 1500 | Cycle 13 | 200 | 200 | 98 | 62 | 62 |
| 15 | 100 | 0 | 1500 | Cycle 14 | 200 | 195 | 98 | 58 | 58 |
| 16 | 100 | 0 | 1500 | Cycle 15 | 200 | 200 | 98 | 65 | 65 |
| 17 | 10 | 0 | 1500 | Cycle 16 | 200 | 210 | 98 | 70 | 70 |
| 18 | 10 | 0 | 1500 | Cycle 17 | 200 | 200 | 98 | 65 | 65 |
| 19 | 100 | 0 | 1500 | Cycle 18 | 200 | 200 | 98 | 66 | 66 |
| 20 | 10 | 0 | 1500 | Cycle 19 | 200 | 200 | 98 | 65 | 65 |
| 21 | 10 | 0 | 1500 | Cycle 20 | 200 | 200 | 98 | 69 | 69 |
| 22 | 10 | 0 | 1500 | Cycle 21 | 200 | 200 | 98 | 65 | 65 |
| 23 | 10 | 0 | 1500 | Cycle 22 | 200 | 5195 | 98 | 68 | 68 |
| 24 | 100 | 0 | 1500 | Cycle 23 | 200 | 195 | 98 | 65 | 65 |
| 25 | 100 | 0 | 1500 | Cycle 24 | 200 | 195 | 98 | 66 | 66 |

### EXAMPLE 4

### 3,5-DI NITRO BENZOIC ACID TO 3,5-DI AMINO BENZOIC ACID

Fresh cycle: In a 2-Liter-4-neck round bottom flask equipped with stirrer, condenser, thermometer, addition port arranged in suitable heating/cooling system was charged 700 ml water, heated to 80° C. Charged 20 ml 50% H2SO4 to get pH 2.0 and immediately charged 60 g G-CAT start up with continuous stirring at 80° C. First lot of 15.3 g Nitro & 20 g G-CAT was charged to the reaction mass in 10 min. The reaction mass was maintained for 5-10 min at 98-100° C. Remaining lots of nitro & G-CAT was charged in four equal lots in similar manner as followed for first lot. Reaction mass was maintained at 98-100° C. for 60 min. Then 160.0 ml 25% Na2CO3 was charged to adjust pH 8.5 in 30 min to adjust pH 8.5. Then it is decanted & collected in separate flask for further processing. 400 ml hot water wash given to solid inorganic by-product, remaining in the flask under stirring at 98°-100° C., to get 335 g of dry cake of solid inorganic by-product & 0.34% amine content, and appearance was black. The decanted mass is then cooled to 30-35° C. and charged 49 ml 50% Sulphuric acid (H2SO4) slowly in 1.0 hrs to get pH 3.5 to 4.0. Liquid layer was then filtered and crystalline material was washed with 150 ml water to get 42 g off white with purity 98.56%.with melting range 234° C. Total filtrate i.e. reaction medium and washings was collected & recycled in subsequent batches.

First recycle: In the same set up as described above, 700 ml reaction medium generated in fresh cycle was charged, heated to 90° C. Charged 20 ml 50% H2SO4 to get pH 2.0 and immediately charged 60 g G-CAT start up with continuous stirring at 90-100° C. First lot of 15.3 g Nitro & 20 g G-CAT was charged to the reaction mass in 10 min. The reaction mass was maintained for 5-10 min at 98-100° C. Remaining lots of nitro & G-CAT was charged in four equal lots in similar manner as followed for first lot. Reaction mass was maintained at 98-100° C. for 60 min. Then 165.0 ml 25% Na2CO3 was charged in 30 min to adjust pH 8.5. Then it is decanted & collected in separate flask for further processing. 400 ml hot water wash given to solid inorganic by-product, remaining in the flask under stirring at 98°-100° C., to get 340 g of dry cake of solid inorganic by-product & 0.34% amine content, and appearance was black. The decanted mass is then cooled to 30-35° C. and charged 58 ml 50% Sulphuric acid (H2SO4) slowly in 1.0 hrs to get pH 3.5 to 4.0. Liquid layer was then filtered and crystalline material was washed with 150 ml water to get 63 g off white with purity 98.26%.with melting range 230° C. Total filtrate i.e. reaction medium and washings was collected & recycled in subsequent batches.

Fifth recycle: In the same set up as described above, 700 ml reaction medium generated in fourth cycle was charged, heated to 90° C. Charged 20 ml 50% H2SO4 to get pH 2.0 and immediately charged 60 g G-CAT start up with continuous stirring at 90-100° C. First lot of 15.3 g Nitro & 20 g G-CAT was charged to the reaction mass in 10 min. The reaction mass was maintained for 5-10 min at 98-100° C. Remaining lots of nitro & G-CAT was charged in four equal lots in similar manner as followed for first lot. Reaction mass was maintained at 98-100° C. for 60 min. Then 170.0 ml 25% Na2CO3 was charged in 30 min to adjust pH 8.5. Then it is decanted & collected in separate flask for further processing. 400 ml hot water wash given to solid inorganic by-product, remaining in the flask under stirring at 98°-100° C., to get 370 g of dry cake of solid inorganic by-product & 0.53% amine content, and appearance was black. The decanted mass is then cooled to 30-35° C. and charged 53 ml 50% Sulphuric acid (H2SO4) slowly in 1.0 hrs to get pH 3.5 to 4.0. Liquid layer was then filtered and crystalline material was washed with 150 ml water to get 70 g off white with purity 97.05%.with melting range 233° C. Total filtrate i.e. reaction medium and washings was collected & recycled in subsequent batches.

Tenth recycle: In the same set up as described above, 700 ml reaction medium generated in ninth cycle was charged, heated to 90° C. Charged 20 ml 50% H2SO4 to get pH 2.0 and immediately charged 60 g G-CAT start up with continuous stirring at 90-100° C. First lot of 15.3 g Nitro & 20 g G-CAT was charged to the reaction mass in 10 min. The reaction mass was maintained for 5-10 min at 98-100° C. Remaining lots of nitro & G-CAT was charged in four equal lots in similar manner as followed for first lot. Reaction mass was maintained at 98-100° C. for 60 min. Then 180.0 ml 25% Na2CO3 was charged in 30 min to adjust pH 8.5. Then it is decanted & collected in separate flask for further processing. 400 ml hot water wash given to solid inorganic by-product, remaining in the flask under stirring at 98°-100° C., to get 340 g of dry cake of solid inorganic by-product & 0.65% amine content, and appearance was black. The decanted mass is then cooled to 30-35° C. and charged 56 ml 50% Sulphuric acid (H2SO4) slowly in 1.0 hrs to get pH 3.5 to 4.0. Liquid layer was then filtered and crystalline material was washed with 150 ml water to get 67 g off white with purity 92.65% with melting range 230° C. Total filtrate i.e. reaction medium and washings was collected & recycled in subsequent batches.

Fifteenth recycle: In the same set up as described above, 700 ml reaction medium generated in fourteen Recycle was charged, heated to 90° C. Charged 20 ml 50% H2SO4 to get pH 2.0 and immediately charged 60 g G-CAT start up with continuous stirring at 90-100° C. First lot of 15.3 g Nitro & 20 g G-CAT was charged to the reaction mass in 10 min. The reaction mass was maintained for 5-10 min at 98-100° C. Remaining lots of nitro & G-CAT was charged in four equal lots in similar manner as followed for first lot. Reaction mass was maintained at 98-100° C. for 60 min. Then 195.0 ml 25% Na2CO3 was charged in 30 min to adjust pH 8.5. Then it is decanted & collected in separate flask for further processing. 400 ml hot water wash given to solid inorganic by-product, remaining in the flask under stirring at 98°-100° C., to get 330 g of dry cake of solid inorganic by-product & 0.66% amine content, and appearance was black. The decanted mass is then cooled to 30-35° C. and charged 59 ml 50% Sulphuric acid (H2SO4) slowly in 1.0 hrs to get pH 3.5 to 4.0. Liquid layer was then filtered and crystalline material was washed with 150 ml water to get 65 g off white with purity 95% with melting range 232° C. Total filtrate i.e. reaction medium and washings was collected & recycled in subsequent batches.

Twentieth recycle: In the same set up as described above, 700 ml reaction medium generated in nineteen cycles was charged, heated to 90° C. Charged 20 ml 50% H2SO4 to get pH 2.0 and immediately charged 60 g G-CAT start up with continuous stirring at 90-100° C. First lot of 15.3 g Nitro & 20 g G-CAT was charged to the reaction mass in 10 min. The reaction mass was maintained for 5-10 min at 98-100° C. Remaining lots of nitro & G-CAT was charged in four equal lots in similar manner as followed for first lot. Reaction mass was maintained at 98-100° C. for 60 min. Then 200.0 ml 25% Na2CO3 was charged in 30 min to adjust pH 8.5. Then it is decanted & collected in separate flask for further processing. 400 ml hot water wash given to solid inorganic by-product, remaining in the flask under stirring at 98°-100° C., to get 315 g of dry cake of solid inorganic by-product & 0.68% amine content, and appearance was black. The decanted mass is then cooled to 30-35° C. and charged 63 ml 50% Sulphuric acid (H2SO4) slowly in 1.0 hrs to get pH 3.5 to 4.0. Liquid layer was then filtered and crystalline material was washed with 150 ml water to get 65 g off white with purity 98% with melting range 230° C. Total filtrate i.e. reaction medium and washings was collected & recycled in subsequent batches.

Twenty-Fifth recycle: In the same set up as described above, 700 ml reaction medium generated in twenty-fourth cycles was charged, heated to 90° C. Charged 20 ml 50% H2SO4 to get pH 2.0 and immediately charged 60 g G-CAT start up with continuous stirring at 90-100° C. First lot of 15.3 g Nitro & 20 g G-CAT was charged to the reaction mass in 10 min. The reaction mass was maintained for 5-10 min at 98-100° C. Remaining lots of nitro & G-CAT was charged in four equal lots in similar manner as followed for first lot. Reaction mass was maintained at 98-100° C. for 60 min. Then 195.0 ml 25% Na2CO3 was charged in 30 min to adjust pH 8.5. Then it is decanted & collected in separate flask for further processing. 400 ml hot water wash given to solid inorganic by-product, remaining in the flask under stirring at 98°-100° C., to get 330 g of dry cake of solid inorganic by-product & 0.53% amine content, and appearance was black. The decanted mass is then cooled to 30-35° C. and charged 68 ml 50% Sulphuric acid (H2SO4) slowly in 1.0 hrs to get pH 3.5 to 4.0. Liquid layer was then filtered and crystalline material was washed with 150 ml water to get 66 g off white with purity 97.12% with melting range 231° C. Total filtrate i.e. reaction medium and washings was collected & recycled in subsequent batches.

The following table (Table 05) illustrates the savings in the various quantities of fresh water used and mother liquor recycled in the reaction of the present invention of 5-amino-salicylic acid.

| | | H2O & ML | | | G-CAT & | | | | |
|---|---|---|---|---|---|---|---|---|---|
| | | consumption & saving | | | R-CAT | | | | |
| | Basis | | Treated | | consumption | | Reduction | Dry | |
| | [1] | | mother liquor | | G-CAT | R-CAT | Temp | Product | Yield |
| Cycle | g | H2O | ML | From | g | g | ° c. | (g) | % |
| 0 | 25 | 500 | 0 | 0 | 37.505 | 5 | 95 | 16.7 | 66.8 |
| 1 | 25 | 20 | 480 | Cycle 0 | 37.505 | 5 | 95 | 17.3 | 69.2 |
| 2 | 25 | 10 | 490 | Cycle 1 | 37.505 | 5 | 95 | 18.4 | 73.6 |
| 3 | 25 | 50 | 450 | Cycle 2 | 37.505 | 5 | 95 | 17.2 | 68.8 |
| 4 | 25 | 10 | 490 | Cycle 3 | 37.505 | 5 | 95 | 22.9 | 91.6 |
| 5 | 25 | 25 | 475 | Cycle 4 | 37.505 | 5 | 95 | 17.7 | 70.8 |

### EXAMPLE 5

### 5 nitro salicylic acid to 5-amino salicylic acid

Fresh cycle: In a round bottom flask equipped with stirrer, condenser, thermometer, addition port arranged in suitable heating/cooling system was charged 250 ml water, heated to 95° C. Charge 28.125 g G-CAT start up with continuous stirring at 95° C. First lot of 1.34 g G-CAT and first lot of 3.571 g Nitro and 8.9 ml water was charged to the reaction mass in 20 min. The reaction mass was maintained for 5-10 min at 95° C. Remaining G-CAT & Nitro was charged in six equal lots in similar manner as followed for first lot. Reaction mass was maintained at 98° C. for 30 min. Then 75 ml of water was added and reaction was maintained at 98° C. for 30 min. 5.0 g R-Cat was charged during 30 min Then add 12.5 ml of 50% NaOH in 1 hr at 85-90° C. in 1 hr to adjust pH of reaction mass to 11.5 to 12 and maintain for 30 minutes. Then settle the batch for 45 minutes and decant it. Add 75 ml alkaline water and maintain the mass at 95° C. for 30 minutes and filter the batch. The spent catalyst was washed with 37.5 ml of alkaline water. Collect decan, filtrate and wash layers together and to this add 1.25 g hydrose and 26 ml of 20% H₂SO₄ at 50° C. in 3 hrs. Cool the reaction mass to ambient temperature and maintained for 2 hours; crystalline material was filtered, to get on drying 16.7 g of grey powder colour with purity 98.83%. Total filtrate i.e. reaction medium and washings was collected & recycled in subsequent batches. Total filtrate i.e. reaction medium and washings was collected & recycled in subsequent batches after treatment.

First recycle: In the same set up as described above, 250 ml reaction medium generated in fresh cycle, heated to 95° C. Charge 28.125 g G-CAT start up with continuous stirring at 95° C. First lot of 1.34 g G-CAT and first lot of 3.571 g Nitro and 8.9 ml reaction medium was charged to the reaction mass in 20 min. The reaction mass was maintained for 5-10 min at 95° C. Remaining G-CAT & Nitro was charged in four equal lots in similar manner as followed for first lot. Reaction mass was maintained at 98° C. for 30 min. Then 75 ml of reaction medium was added and reaction was maintained at 98° C. for 30 min 5.0 g R-Cat was charged during 30 min. Then add 12.5 ml of 50% NaOH in 1 hr at 85-90° C. in 1 hr to adjust pH of reaction mass to 11.5 to 12 and maintain for 30 minutes. Then settle the batch for 45 minutes and decant it. Add 75 ml reaction medium and maintain the mass in alkaline condition at 95° C. for 30 minutes and filter the batch. The spent catalyst was washed with mixture of 17.5 ml of reaction medium and 20 ml fresh alkaline water. Collect decant, filtrate and wash layers together and to this add 1.25 g hydrose and 26 ml of 20% H₂SO₄ at 50° C. in 3 hrs. Cool the reaction mass to ambient temperature and maintained for 2 hours; crystalline material was filtered, to get on drying 17.3 g of grey powder colour with purity 99.33%. Total filtrate i.e. reaction medium and washings was collected & recycled in subsequent batches.

Second recycle: In the same set up as described above, 250 ml reaction medium generated in first cycle, heated to 95° C. Charge 28.125 g G-CAT start up with continuous stirring at 95° C. First lot of 1.34 g G-CAT and first lot of 3.571 g Nitro and 8.9 ml reaction medium was charged to the reaction mass in 20 min. The reaction mass was maintained for 5-10 min at 95° C. Remaining G-CAT & Nitro was charged in four equal lots in similar manner as followed for first lot. Reaction mass was maintained at 98° C. for 30 min. Then 75 ml of reaction medium was added and reaction was maintained at 98° C. for 30 min 5.0 g R-Cat was charged during 30 min. Then add 12.5 ml of 50% NaOH in 1 hr at 85-90° C. in 1 hr to adjust pH of reaction mass to 11.5 to 12 and maintain for 30 minutes. Then settle the batch for 45 minutes and decant it. Add 75 ml reaction medium and maintain the mass in alkaline condition at 95° C. for 30 minutes and filter the batch. The spent catalyst was washed with the mixture of 27.5 ml of reaction medium & 10 ml alkaline water. Collect decant, filtrate and wash layers together and to this add 1.25 g hydrose and 26 ml of 20% H₂SO₄ at 50° C. in 3 hrs. Cool the reaction mass to ambient temperature and maintained for 2 hours; crystalline material was filtered, to get on drying 18.4 g of grey powder colour with purity 98.77%. Total filtrate i.e. reaction medium and washings was collected & recycled in subsequent batches.

Third recycle: In the same set up as described above, 250 ml reaction medium generated in second cycle, heated to 95° C. Charge 28.125 g G-CAT start up with continuous stirring at 95° C. First lot of 1.34 g G-CAT and first lot of 3.571 g Nitro and 8.9 ml reaction medium was charged to the reaction mass in 20 min The reaction mass was maintained for 5-10 min at 95° C. Remaining G-CAT & Nitro was charged in four equal lots in similar manner as followed for first lot. Reaction mass was maintained at 98° C. for 30 min. Then 75 ml of reaction medium was added and reaction was maintained at 98° C. for 30 min 5.0 g R-Cat was charged during 30 min. Then add 12.5 ml of 50% NaOH in 1 hr at 85-90° C. in 1 hr to adjust pH of reaction mass to 11.5 to 12 and maintain for 30 minutes. Then settle the batch for 45 minutes and decant it. Add 62.5 ml reaction medium, 12.5 ml fresh alkaline water and maintain the mass in alkaline condition at 95° C. for 30 minutes and filter the batch. The spent catalyst was washed with 37.5 ml of fresh alkaline water. Collect decan, filtrate and wash layers together and to this add 1.25 g hydrose and 26 ml of 20% H₂SO₄ at 50° C. in 3 hrs. Cool the reaction mass to ambient temperature and maintained for 2 hours; crystalline material was filtered, to get on drying 17.2 g of grey powder colour with purity 98.99%. Total filtrate i.e. reaction medium and washings was collected & recycled in subsequent batches. Total filtrate i.e. reaction medium and washings was collected & recycled in subsequent batches after treatment.

Fourth recycle: In the same set up as described above, 250 ml reaction medium generated in third cycle, heated to 95° C. Charge 28.125 g G-CAT start up with continuous stirring at 95° C. First lot of 1.34 g G-CAT and first lot of 3.571 g Nitro and 8.9 ml reaction medium was charged to the reaction mass in 20 min. The reaction mass was maintained for 5-10 min at 95° C. Remaining G-CAT & Nitro was charged in four equal lots in similar manner as followed for first lot. Reaction mass was maintained at 98° C. for 30 min. Then 75 ml of reaction medium was added and reaction was maintained at 98° C. for 30 min. 5.0 g R-Cat was charged during 30 min. Then add 12.5 ml of 50% NaOH in 1 hr at 85-90° C. in 1 hr to adjust pH of reaction mass to 11.5 to 12 and maintain for 30 minutes. Then settle the batch for 45 minutes and decant it. Add 75 ml reaction medium and maintain the mass in alkaline condition at 95° C. for 30 minutes and filter the batch. The spent catalyst was washed with the mixture of 27.5 ml of reaction medium and 10 ml of fresh alkaline water. Collect decant, filtrate and wash layers together and to this add 1.25 g hydrose and 26 ml of 20% H₂SO₄ at 50° C. in 3 hrs. Cool the reaction mass to ambient temperature and maintained for 2 hours; crystalline material was filtered, to get on drying 22.9 g of bluish Violet powder with purity 97.65%. Total filtrate i.e. reaction medium and washings was collected & recycled in subsequent batches.

Fifth recycle: In the same set up as described above, 250 ml reaction medium generated in fourth cycle, heated to 95° C. Charge 28.125 g G-CAT start up with continuous stirring at 95° C. First lot of 1.34 g G-CAT and first lot of 3.571 g Nitro and 8.9 ml reaction medium was charged to the reaction mass in 20 min. The reaction mass was maintained for 5-10 min at 95° C. Remaining G-CAT & Nitro was charged in four equal lots in similar manner as followed for first lot. Reaction mass was maintained at 98° C. for 30 min. Then 75 ml of reaction medium was added and reaction was maintained at 98° C. for 30 min 5.0 g R-Cat was charged during 30 min. Then add 12.5 ml of 50% NaOH in 1 hr at 85-90° C. in 1 hr to adjust pH of reaction mass to 11.5 to 12 and maintain for 30 minutes. Then settle the batch for 45 minutes and decant it. Add 75 ml reaction medium and maintain the mass in alkaline condition at 95° C. for 30 minutes and filter the batch. The spent catalyst was washed with the mixture of 25 ml reaction medium and 12.5 ml of alkaline water. Collect decant, filtrate and wash layers together and to this add 1.25 g hydrose and 26 ml of 20% H₂SO₄ at 50° C. in 3 hrs. Cool the reaction mass to ambient temperature and maintained for 2 hours; crystalline material was filtered, to get on drying 17.7 g of grey powder colour with purity 98.16%. Total filtrate i.e. reaction medium and washings was collected & recycled in subsequent batches. Total filtrate i.e. reaction medium and washings was collected & recycled in subsequent batches after treatment.

The following table (Table 06) illustrates the savings in the various quantities of fresh water used and mother liquor recycled in the reaction of the present invention of DNDS to DADS.

| | | | | | Fresh Solvent consumption & | | | | |
|---|---|---|---|---|---|---|---|---|---|
| | | H2O & ML | | | saving by solvent recycle | | | | |
| | | consumption & | | | | | Quantity | | |
| | | saving | | | | Recovered | (ml) of | | |
| | Basis | | Treated | | Fresh | from | Solvent | Dry | |
| | [1] | | mother liquor | | solvent | Cycle | Recycle | Product | Yield |
| Cycle | gm | H2O | ML | From | ml | No. | ml | gm | % |
| 0 | 100 | 1250 | 0 | Cycle 0 | 970 | 0 | 0 | 20 | 20 |
| 1 | 100 | 250 | 1000 | Cycle 0 | 194 | Cycle 0 | 776 | 145 | 145 |
| 2 | 100 | 250 | 1000 | Cycle 1 | 194 | Cycle 1 | 776 | 42 | 42 |
| 3 | 100 | 250 | 1000 | Cycle 2 | 194 | Cycle 2 | 776 | 42 | 42 |
| 4 | 100 | 250 | 1000 | Cycle 3 | 194 | Cycle 3 | 776 | 90 | 90 |
| 5 | 100 | 250 | 1000 | Cycle 4 | 194 | Cycle 4 | 776 | 131 | 131 |

### Experimental Procedure for-DNDS TO DADS

Fresh cycle: In a round bottom flask equipped with stirrer, condenser, thermometer, addition port arranged in suitable heating/cooling system was charged 500 ml water, heated to 100° C. Charged 20% H2SO4 to get pH 4.0 and 50 g NRC start up with continuous stirring at 100° C. First lot of 10 g NRC and first lot of 11.2 g Nitro was charged to the reaction mass in 20 min. The reaction mass was maintained for 20 min at 100° C. Remaining NRC & Nitro was charged in four equal lots in similar manner as followed for first lot. NGC was charged to adjust pH of reaction mass to 7.0 and maintain for 15 minutes. Then filter whole batch take completely dry grinded spent charged 150 ml DMF, heated to 80° c. Stir it for 2 hr then it is decanted and collected in separate flask for further processing. Charged 150 ml DMF was charged for extraction, stir it for 2 hr at 80° C. and stirring was stopped and upper liquid layer was decanted to the crystallizer. Again 150 ml DMF was added to solid inorganic by-product, remaining in the flask under stirring at 80° C. for 2 hr. Then filter it give wash of 25 ml hot DMF. Collect all decanted mass & washing. Distill out approx. 60-70% from total volume of DMF at 80-85° C. in vacuum distillation. Chill distillate up to 0-5° c, crystalline material was filtered, to get on drying 20 g Off white powder with purity 99.39%. Mother liquor was recycled in subsequent batches.

First Recycle (R1): In the same set up as described above, 500 ml water, heated to 100° C. Charged 20% H2SO4 to get pH 4.0 and 50 g NRC start up with continuous stirring at 100° C. First lot of 10 g NRC and first lot of 11.2 g Nitro was charged to the reaction mass in 20 min. The reaction mass was maintained for 20 min at 100° C. Remaining NRC & Nitro was charged in four equal lots in similar manner as followed for first lot. NGC was charged to adjust pH of reaction mass to 7.0 and maintain for 15 minutes. Then filter whole batch take completely dry grinded spent charged 150 ml DMF, heated to 80° c. Stir it for 2 hr then it is decanted and collected in separate flask for further processing. Charged 150 ml DMF was charged for extraction, stir it for 2 hr at 80° C. and stirring was stopped and upper liquid layer was decanted to the crystallizer. Again 150 ml DMF was added to solid inorganic by-product, remaining in the flask under stirring at 80° C. for 2 hr. Then filter it give wash of 25 ml hot DMF. Collect all decanted mass & washing. Distill out approx. 60-70% from total volume of DMF at 80-85° c in vacuum distillation. Chill distillate up to 0-5° c, crystalline material was filtered, to get on drying 145 g Off white powder with purity 99.30%. Mother liquor was recycled in subsequent batches.

Second Recycle (R2): In the same set up as described above, 500 ml water, heated to 100° C. Charged 20% H2SO4 to get pH 4.0 and 50 g NRC start up with continuous stirring at 100° C. First lot of 10 g NRC and first lot of 11.2 g Nitro was charged to the reaction mass in 20 min. The reaction mass was maintained for 20 min at 100° C. Remaining NRC & Nitro was charged in four equal lots in similar manner as followed for first lot. NGC was charged to adjust pH of reaction mass to 7.0 and maintain for 15 minutes. Then filter whole batch take completely dry grinded spent charged 150 ml DMF, heated to 80° c. Stir it for 2 hr then it is decanted and collected in separate flask for further processing. Charged 150 ml DMF was charged for extraction, stir it for 2 hr at 80° C. and stirring was stopped and upper liquid layer was decanted to the crystallizer. Again 150 ml DMF was added to solid inorganic by-product, remaining in the flask under stirring at 80° C. for 2 hr. Then filter it give wash of 25 ml hot DMF. Collect all decanted mass & washing. Distill out approx. 60-70% from total volume of DMF at 80-85° c in vacuum distillation. Chill distillate up to 0-5° c, crystalline material was filtered, to get on drying 42 g Off white powder with purity 99.30%. Mother liquor was recycled in subsequent batches.

Third Recycle (R3): In the same set up as described above, 500 ml water, heated to 100° C. Charged 20% H2SO4 to get pH 4.0 and 50 g NRC start up with continuous stirring at 100° C. First lot of 10 g NRC and first lot of 11.2 g Nitro was charged to the reaction mass in 20 min. The reaction mass was maintained for 20 min at 100° C. Remaining NRC & Nitro was charged in four equal lots in similar manner as followed for first lot. NGC was charged to adjust pH of reaction mass to 7.0 and maintain for 15 minutes. Then filter whole batch take completely dry grinded spent charged 150 ml DMF, heated to 80° c. Stir it for 2 hr then it is decanted and collected in separate flask for further processing. Charged 150 ml DMF was charged for extraction, stir it for 2 hr at 80° C. and stirring was stopped and upper liquid layer was decanted to the crystallizer. Again 150 ml DMF was added to solid inorganic by-product, remaining in the flask under stirring at 80° C. for 2 hr. Then filter it give wash of 25 ml hot DMF. Collect all decanted mass & washing. Distill out approx. 60-70% from total volume of DMF at 80-85° c in vacuum distillation. Chill distillate up to 0-5° c, crystalline material was filtered, to get on drying 42 g Off white powder with purity 99.39%. Mother liquor was recycled in subsequent batches.

Fourth Recycle (R4): In the same set up as described above, 500 ml water, heated to 100° C. Charged 20% H2SO4 to get pH 4.0 and 50 g NRC start up with continuous stirring at 100° C. First lot of 10 g NRC and first lot of 11.2 g Nitro was charged to the reaction mass in 20 min. The reaction mass was maintained for 20 min at 100° C. Remaining NRC & Nitro was charged in four equal lots in similar manner as followed for first lot. NGC was charged to adjust pH of reaction mass to 7.0 and maintain for 15 minutes. Then filter whole batch take completely dry grinded spent charged 150 ml DMF, heated to 80° c. Stir it for 2 hr then it is decanted and collected in separate flask for further processing. Charged 150 ml DMF was charged for extraction, stir it for 2 hr at 80° C. and stirring was stopped and upper liquid layer was decanted to the crystallizer. Again 150 ml DMF was added to solid inorganic by-product, remaining in the flask under stirring at 80° C. for 2 hr. Then filter it give wash of 25 ml hot DMF. Collect all decanted mass & washing. Distill out approx. 60-70% from total volume of DMF at 80-85° c in vacuum distillation. Chill distillate up to 0-5° c, crystalline material was filtered, to get on drying 90 g Off white powder with purity 99.39%. Mother liquor was recycled in subsequent batches.

Fifth Recycle (R5): In the same set up as described above, 500 ml water, heated to 100° C. Charged 20% H2SO4 to get pH 4.0 and 50 g NRC start up with continuous stirring at 100° C. First lot of 10 g NRC and first lot of 11.2 g Nitro was charged to the reaction mass in 20 min. The reaction mass was maintained for 20 min at 100° C. Remaining NRC & Nitro was charged in four equal lots in similar manner as followed for first lot. NGC was charged to adjust pH of reaction mass to 7.0 and maintain for 15 minutes. Then filter whole batch take completely dry grinded spent charged 150 ml DMF, heated to 80° c. Stir it for 2 hr then it is decanted and collected in separate flask for further processing. Charged 150 ml DMF was charged for extraction, stir it for 2 hr at 80° C. and stirring was stopped and upper liquid layer was decanted to the crystallizer. Again 150 ml DMF was added to solid inorganic by-product, remaining in the flask under stirring at 80° C. for 2 hr. Then filter it give wash of 25 ml hot DMF. Collect all decanted mass & washing. Distill out approx. 60-70% from total volume of DMF at 80-85° c in vacuum distillation. Chill distillate up to 0-5° c, crystalline material was filtered, to get on drying 131 g off white powder with purity 99.30%. Mother liquor was recycled in subsequent batches.

The following table (Table 07) illustrates the savings in the various quantities of fresh water used and mother liquor recycled in the reaction of the present invention of 4-Chloro 2-Nitro Phenol to 4-Chloro 2-Amino Phenol (4-CAP).

| | H2O & ML | | | |
|---|---|---|---|---|
| | consumption & saving | | | |
| Basis | | Treated | Dry | |
| [1] | | mother liquor | Product | Yield |

| Cycle | gm | H2O | ML | From | gm | % |
|---|---|---|---|---|---|---|
| 0 | 100 | 600 | 0 | Cycle 0 | 61 | 61 |

### Experimental Procedure for 4-Chloro-2-NitroPhenol to 4-Chloro-2-Amino Phenol

Fresh cycle: In a round bottom flask equipped with stirrer, condenser, thermometer, addition port arranged in suitable heating/cooling system was charged 400 ml water, heated to 100° C. Charged 20% diluted H2SO4 to get pH 4.5 and 40 g NRC start up with continuous stirring at 100° C. First lot of 20 g NRC and first lot of 20 g Nitro was charged to the reaction mass in 30 min. The reaction mass was maintained for 15 min at 100° C. Remaining NRC & Nitro was charged in four equal lots in similar manner as followed for first lot. Reaction mass was maintained at 100° C. for 30 min. NGC was charged during 30 min to adjust pH of reaction mass to 7.0 and maintain for 15 minutes. Na2CO3 was charged to get pH 12.0 & maintain for 30 min at 100° C. Then filter at hot condition & use 200ml hot alkaline water for spent add Hydrose during filtration in filtration flask. Collect the filtrate & get pH 1.0 by diluted HCL at 70° c. Add 10 g plant carbon with continuous stirring & add pinch of Hydrose to maintain colour of the filtrate & stirred for 30 min at 90° c, filter the filtrate again. Collect the filtrate & get pH 5.0 by 45% NaOH solution at 65° c. Liquid layer in the crystallizer was cooled to 5 to 10° c, crystalline material was filtered to get on drying 61 g of white powder colour with purity 95%.

The following table (Table 08) illustrates the savings in the quantities of various fresh water used in the reaction of the present invention of ADPSA.

| | | H2O & ML | | | | | | | |
|---|---|---|---|---|---|---|---|---|---|
| | | consumption & saving | | | NRC & NPC | | | | |
| | Basis | | Treated | | consumption | | Reduction | Dry | |
| | [1] | | mother liquor | | NRC | NGC | Temp | wt | Yield |
| Cycle | g | H2O | ML | From | gm | gm | ° c. | gm | % |
| 0 | 50 | 400 | 0 | \acute{ } | 32.5 | 5.0 | 98-100 | 41 | 82 |

### Experimental Procedure for 4-NDPSA to 4-ADPSA

First cycle: In the same set up as described above, charged 125 ml water, heated to 95° C. Charged 20% H2SO4 to get pH 4.0 and 17.5 g NRC start up with continuous stirring at 95° C. First lot of 3.0 g NRC and first lot of 11 g Nitro was charged to the reaction mass in 10 min. The reaction mass was maintained for 10 min at 98-100° C. Remaining NRC & Nitro was charged in four equal lots in similar manner as followed for first lot. NGC was charged during 30 min to adjust pH of reaction mass to 8.5 and maintain for 15 minutes. Then filter whole batch through Buckner funnel add 1 g hydrose during filtration. Give 100 ml hot alkaline water wash to NRC-spent (1^{st} wash) keep it separate. Again give 125 ml hot alkaline water wash to NRC-spent (2^{nd} wash) keep it separate. Again give 50 ml hot alkaline water wash to NRC-spent (3^{rd} wash) keep it separate. Then take filtrate & 1^{st} wash together for isolation. Rise the temperature up to 70-80° c. Charged 32% diluted Sulphuric acid slowly in 2 hrs at pH Congo +ve. Liquid layer in the crystallizer was chilling at 10-15° c with stirring for 1 hours; crystalline material was filtered, to get on drying 41 g of faint grey powder with purity 40.23%. Total filtrate i.e. reaction medium and washings was collected & recycled in subsequent batches.

The following table (Table 09) illustrates the savings in the quantities of various fresh water used in the reaction of the present invention of OAPSA

| | | H2O & ML | | | NRC & NPC | | | | |
|---|---|---|---|---|---|---|---|---|---|
| | | consumption & saving | | | consumption | | | | |
| | Basis | | Treated | | | NGC gm/ | Reduction | Dry | |
| | [1] | | mother liquor | | NRC | Caustic lye | Temp | wt | Yield |
| Cycle | g | H2O | ML | From | gm | ml | ° c. | gm | % |
| 0 | 70 | 450 | 0 | \acute{ } | 23 | 12.0 100 | 98-100 | 47.6 | 68 |
| 1 | 70 | 240 | 210 | Cycle 0 | 23 | 10.0 110 | 98-100 | 53.2 | 76 |
| 2 | 70 | 280 | 170 | Cycle 1 | 23 | 10.5 108 | 98-100 | 53.2 | 76 |
| 3 | 70 | 270 | 180 | Cycle 2 | 23 | 12.0 115 | 98-100 | 53.2 | 76 |

### Experimental Procedure for-ONPSA TO OAPSA

Fresh cycle: In a round bottom flask equipped with stirrer, condenser, thermometer, addition port arranged in suitable heating/cooling system was charged 200 ml water, heated to 98° C. Charged 6 ml diluted HCL to get pH 2.0 and 20 g NRC start up with continuous stirring at 98° C. First lot of 5 g NRC and first lot of 14 g Nitro was charged to the reaction mass in 15 min. Add 20 ml water per lot. The reaction mass was maintained for 5 min at 98° C. Remaining G-CAT & Nitro was charged in four equal lots in similar manner as followed for first lot. NGC-HP-01 was charged during 15 min to adjust pH of reaction mass to 7.0 and then add 45% Caustic lye solution to make pH 10.5 then maintain 15 min and filter the whole mass Buckner funnel and give the washing 150 ml hot water to the NRC spent. The final filtrate volume is 335 ml evaporate the volume up to 225 ml during evaporation maintain filtrate colour by hydrose. Add NaCl 10% of volume and charged 30% Sulphuric acid to make pH congo red. Liquid layer in the crystallizer was cooled to 20° c, Crystalline material was filtered and give 100 ml water wash. Dry at 70° c to get on drying 47.6 g of Off white powder with purity 97%.

First cycle: In the same set up as described above was charged 210 ml reaction medium generated in fresh cycle, heated to 98° C. Charged 6 ml dilute HCL to get pH 2.0 and 20 g NRC start up with continuous stirring at 98° C. First lot of 5 g NRC and first lot of 14 g Nitro was charged to the reaction mass in 15 min. Add 20 ml water per lot. The reaction mass was maintained for 5 min at 98° C. Remaining G-CAT & Nitro was charged in four equal lots in similar manner as followed for first lot. NGC-HP-01 was charged during 15 min to adjust pH of reaction mass to 7.0 and then add 45% Caustic lye solution to make pH 10.5 then maintain 15 min and filter the whole mass Buckner funnel and give the washing 150 ml hot water to the NRC spent. The final filtrate volume is 335 ml evaporate the volume up to 225 ml during evaporation maintain filtrate colour by hydrose. Add NaCl 10% of volume and charged 30% Sulphuric acid to make pH congo red. Liquid layer in the crystallizer was cooled to 20° c, Crystalline material was filtered and give 100 ml water wash. Dry at 70° c to get on drying 53.2 g of Off white powder with purity 97%.

Second cycle: In the same set up as described above was charged 170 ml reaction medium generated in first cycle & 30 ml water, heated to 98° C. Charged 6 ml diluted HCL to get pH 2.0 and 20 g NRC start up with continuous stirring at 98° C. First lot of 5 g NRC and first lot of 14 g Nitro was charged to the reaction mass in 15 min Add 20 ml water per lot. The reaction mass was maintained for 5 min at 98° C. Remaining G-CAT & Nitro was charged in four equal lots in similar manner as followed for first lot. NGC-HP-01 was charged during 15 min to adjust pH of reaction mass to 7.0 and then add 45% Caustic lye solution to make pH 10.5 then maintain 15 min and filter the whole mass Buckner funnel and give the washing 150 ml hot water to the NRC spent. The final filtrate volume is 335 ml evaporate the volume up to 225 ml during evaporation maintain filtrate colour by hydrose. Add NaCl 10% of volume and charged 30% Sulphuric acid to make pH congo red. Liquid layer in the crystallizer was cooled to 20° c, Crystalline material was filtered and give 100 ml water wash. Dry at 70° c to get on drying 53.2 g of Off white powder with purity 97%.

Third cycle: In the same set up as described above was charged 180 ml reaction medium generated in second cycle and 20 ml water, heated to 98° C. Charged 6 ml diluted HCL to get pH 2.0 and 20 g NRC start up with continuous stirring at 98° C. First lot of 5 g NRC and first lot of 14 g Nitro was charged to the reaction mass in 15 min Add 20 ml water per lot. The reaction mass was maintained for 5 min at 98° C. Remaining G-CAT & Nitro was charged in four equal lots in similar manner as followed for first lot. NGC-HP-01 was charged during 15 min to adjust pH of reaction mass to 7.0 and then add 45% Caustic lye solution to make pH 10.5 then maintain 15 min and filter the whole mass Buckner funnel and give the washing 150 ml hot water to the NRC spent. The final filtrate volume is 335 ml evaporate the volume up to 225 ml during evaporation maintain filtrate colour by hydrose. Add NaCl 10% of volume and charged 30% Sulphuric acid to make pH congo red. Liquid layer in the crystallizer was cooled to 20° c, Crystalline material was filtered and give 100 ml water wash. Dry at 70° c to get on drying 53.2 g of Off white powder with purity 97%.

Based on the foregoing discussion it is clear that the present invention comprises the following items:
1. A sustainable chemical process of green reduction of nitro-compounds, R—NO₂, or nitroso compounds, R—NO, having Sulphonic Group or Carboxylic Group into corresponding amino-compounds, R—NH₂, comprising a plurality of cycles, each of said cycles comprising a green reaction sequence and a green isolation sequence, wherein said green isolation sequence follows said green reaction sequence.
2. A process as described in item 1, wherein the number of said plurality of cycles is preferably greater than 3, more preferably greater than 25, even more preferably greater than 100.
3. A process as described in any of items 1 and 2,
   Wherein said green reaction sequence of a typical said cycle comprises the following steps:
   Step 1.1—creating start-up conditions for the reduction process, said Step 1.1 further comprising the following stages:
      Stage 1.1a. charging a suitable reaction medium, denoted as start-up reaction medium, to a first reaction vessel with an agitator and other attachments known to a person skilled in the art; thereby forming the start-up reaction mixture;
      wherein the quantity of the reaction medium used in Step 1.1, denoted as Q_{RM1.1}, is variable, said Q_{RM1.1} being in the range of 0% (w/w) to 40% (w/w) of Q_{RMT} used in this cycle;
      wherein said Q_{RMT} is the total quantity of the reaction medium used in this cycle, said Q_{RMT} being determined such that the ratio, denoted as (Weight Ratio)_{RM}, of the weight of said Q_{RMT}, W_{RM}, to the weight of total amount of R—NO₂ or R—NO to be reduced in that single cycle W_{N}; the relationship between (Weight Ratio)_{RM}, W_{RM}, and W_{N} being represented by the equation

(Weight Ratio)_{RM}=W_{RM}/W_{N};
and wherein said (Weight Ratio)_{RM} is preferably in the range of 5 to 100, the more preferable range being 10 to 75, and;
stage 1.1b. further optionally charging to said first reaction vessel a first suitable acid, preferably sulfuric acid, in a suitable quantity, and in suitable form, said suitable form being solid, liquid, or any combination thereof, to said SRM such that the pH of said SRM is in the range between 1 to 9, preferably between 3 to 7, more preferably between 4 to 6; wherein said acid is added at a start-up temperature which is in the range between 0° C. to 200° C.;
stage 1.1c. agitating the mixture thus formed for a duration in the range of 0 minutes to 5 hours, more preferably between 0.5 hours to 2.5 hours, while maintaining the pH of the mixture during the agitation stage of stage 1.1c in the range between 1 to 9, preferably between 3 to 7, more preferably between 4 to 6, and while maintaining the temperature of the mixture during the agitation stage of stage 1.1c in the range between 0° C. to 200° C.; and
stage 1.1d. adding to said first reaction vessel, upon completion of the agitation stage of stage 1.1c, a reducing agent, RA_{1.1} in suitable quantity which is denoted as Q_{RA1.1}, said Q_{RA1.1} being variable in the range of 0% to 100% of Q_{RAT};
wherein said Q_{RAT} is the total quantity of the reducing agent used in this cycle; said Q_{RAT} being determined such that the ratio, denoted as (Weight Ratio)_{RA}, of the weight of said Q_{RAT}, W_{RA}, to the weight of total amount of R—NO₂ or R—NO to be reduced in that single cycle, W_{N}; wherein the relationship between (Weight Ratio)_{RA}, W_{RA}, and W_{N} is represented by the equation:

(Weight Ratio)_{RA}=W_{RA}/W_{N};
and wherein said (Weight Ratio)_{RA} is preferably in the range of 0.25 to 25, the more preferable range being 0.5 to 2.5;
wherein said reducing agent is added either in its full required quantity, Q_{RA1.1}, or in batches, or continuously, or as any combination of these methods of addition, over a period of 0 minutes to 5 hours, preferably 0.5 hours to 2.5 hours; and wherein the pH of the mixture in said first reaction vessel at the time of addition of said reducing agent is between 1 to 9, preferably between 2 to 7;
Step 1.2—reducing the nitro or nitroso compound to be reduced, wherein said Step 1.2 further comprises the following stages:
   stage 1.2a. adding R—NO₂ or R—NO, respectively a nitro or nitroso compound to be reduced, to said reaction vessel over a suitable reduction period in the range of 0 to 25 hours;
   stage 1.2b. charging a suitable reaction medium, denoted as reduction reaction medium, in suitable quantity to said first reaction vessel;
   stage 1.2c. further optionally adding to said first reaction vessel a second suitable acid, preferably sulfuric acid, to bring the pH value of the mixture thus formed, referred to as reduction mixture, with in the range between 1 to 9, preferably between 2 to 7, more preferably between 4 to 6, while maintaining the temperature of the mixture formed by addition the acid to said reduction mixture between 0° C. to 200° C.; and
   stage 1.2d. adding a reducing agent, RA_{1.2}, to said first reaction vessel, wherein said RA_{1.2} is added either simultaneously with the R—NO₂ or R—NO compound to be reduced in stage 1.2a, or after the addition of acid of stage 1.2c, thereby forming a reduction agent mixture; wherein said RA_{1.2} is added at a reduction time such that the pH of said reduction agent mixture is in a range between 1 to 9, preferably between 2 to 7, more preferably between 4 to 6, and such that the temperature of said reduction mixture is in the range between 0° C. to 200° C.;

Wherein the quantity of RA_{1.2}, denoted as Q_{R1.2}, is such that said Q_{R1.2} is the difference between Q_{RT} and Q_{R1.1};
Step 1.3—neutralizing the reaction mixture obtained at the end of Step 1.2, wherein said neutralization is carried out in the following stages:
   stage 1.3a. optionally adding a suitable reaction medium, denoted as neutralization reaction medium, in a suitable quantity to said reaction vessel, wherein quantity of the reaction medium used in Step 1.3, denoted as Q_{RM1.3}, is variable in the range of 0% (w/w) to 40% (w/w) of Q_{RMT};
   stage 1.3b. adding to the reaction mixture obtained at the end of step 1.3a in said first reaction vessel a neutralizing agent, NA_{1.3}, wherein the quantity of said neutralizing agent used, Q_{NAT}, is the total quantity of the neutralizing agent to be used in this cycle; said Q_{NAT} being determined such that the ratio, denoted as (Weight Ratio)_{NA}, of the weight of said Q_{NAT}, W_{NA}, to the weight of total amount of R—NO₂ or R—NO to be reduced in that single cycle, W_{N}; wherein the relationship between (Weight Ratio)_{NA}, W_{NA}, and W_{N} is represented by the equation:

(Weight Ratio)_{NA}=W_{NA}/W_{N};
and wherein said (Weight Ratio)_{NA} is preferably in the range of 0 to 2.5, the more preferable range being 0.05 to 0.25; and
wherein said neutralizing agent is added over a period in the range between 0 minutes to 5 hours, more preferably between 0.5 hours to 2.5 hours, at a temperature in the range between 0° C. to 200° C., and at a pH in the range between 1 to 9, preferably 2 to 8;
wherein R-Cat or G-Cat is used as the preferred neutralizing agent; and
stage 1.3c. allowing the neutralization of the mixture obtained at the end of stage 1.3b containing R—NO₂ or R—NO compound to take place at a temperature between 0° C. to 200° C., at a pH between 1 to 9, preferably 2 to 8, said neutralization being carried out over a period between 0 hours to 10 hours, preferably in the range of 30 minutes to 5 hours;
wherein the contents of said first reaction vessel during any or all stages of 1.3a to 1.3c are optionally stirred for any duration of the individual stages using said agitator rotating at a rate between 0 to 500 RPM;
Step 1.4—Isolating the appropriate part of the contents of the said first reaction vessel obtained at the end of Step 1.3, wherein the isolation process comprises the following stages:
   stage 1.4a. charging to the reaction mixture obtained at the end of stage 1.3c, G-Cat, wherein the quantity of said G-Cat being such that its weight ratio with R—NO₂ or R—NO is in the range of a 0.05 w/w to 5 w/w, preferable range being 0.5 w/w to 2.5 w/w, thereby forming an Isolation mixture, at a time such that the pH of said isolation reaction mixture is in the range of 1 to 12, preferably 4 to 11;
   stage 1.4b. optionally adding a suitable reaction medium, denoted as isolation reaction medium, after completion of or any time during stage 1.4a, at a temperature between 0° C. and 200° C., and at pH level between the range of 1 to 12 preferably 4 to 11; and
   stage 1.4c. maintaining the isolation mixture obtained at the end of stage 1.4b at a temperature between 0° C. and 200° C., preferably between 0° C. to 100° C., for a period in the range of 0 hours to 24 hours, preferably in the range of 30 minutes to 5 hours;
whereby a single cycle of said green reaction sequence is completed, and where after a cycle of green isolation sequence is carried out, said green isolation sequence comprising the following steps:
Step 2.1—applying settling and decantation to the contents of said reaction vessel obtained at the end of said step 1.4, wherein said settling and decantation comprises following stages:
   stage 2.1a. optionally charging a suitable reaction medium, denoted as first settling reaction medium, to the reaction mixture obtained at the end of step 1.4, maintaining the temperature of the mixture in the range between 0° C. and 200° C., and the pH of the mixture in the range between 3 to 14, preferably between 4 to 12; wherein the quantity of said first settling reaction medium used, denoted as Q_{RM2.1}, is variable in the range of 0% (w/w) to 60% (w/w) of the Q_{RMT} used in this cycle.
   stage 2.1b. allowing the reaction mixture obtained at the end of stage 2.1b to settle down for a first settling time while maintaining the temperature of the mixture in the range between 0° C. and 200° C., and the pH of the mixture in the range between 3 to 14, preferably between 4 to 12; wherein said first settling time is in the range between 1 minute to 10 hours, preferably between 30 minutes to 3 hours;
   stage 2.1c. decanting the liquid layer formed at the end of stage 2.1c at a first decanting temperature in the range between 0° C. and 200° C., a first decanting pH in the range between 3 to 14, preferably between 4 to 12; and first decanting time in the range between 1 minute to 10 hours, preferably between 30 minutes to 3 hours, and charging the decanted liquid Stream A to Step 2.5 of same cycle or any of the following cycles;
Step 2.2—stirring, settling, and decanting the contents obtained at the end of Step 2.1, the stirring, settling and decanting comprising the following stages:
   stage 2.2a. charging to said reaction vessel a suitable reaction medium, denoted as second settling reaction medium, at a predetermined first stirring temperature and a predetermined first stirring pH at a predetermined first stirring time;
   stage 2.2b. stirring and continuing to stir the mixture of stage 2.2a by maintaining the mixture at a predetermined first stirring continuation temperature, a predetermined first stirring continuation pH for a predetermined first stirring continuation time;
   stage 2.2c. stopping the stirring action and allowing the mixture of stage 2.2b to settle at a predetermined second settling pH, a predetermined second settling temperature for a predetermined second settling time; and
   stage 2.2d. decanting the liquid layer collected at the end of stage 2.2c, the liquid layer denoted as Stream B, at a predetermined second decantation temperature, a predetermined second decantation pH and at a predetermined second decantation time; said Stream B being charged to Step 2.5 of the same cycle or any of the following cycles;
   wherein the values each of said first stirring temperature, said first stirring continuation temperature, and said second decantation temperature are in the range of 0° C. and 200° C., preferably between 0° C. to 100° C.; the values of each of said first stirring pH, said first stirring continuation pH, and said second decantation pH are in the range of between 3 to 14, preferably between 4 to 12; the values of each of said first stirring time, said first stirring continuation time, and said second decantation time are in the range of 5 minutes to 5 hours; preferably 30 minutes to 3 hours; and
   wherein the quantity of the reaction medium used in Step 2.2, denoted as Q_{RM2.2} is variable in the range of 0% (w/w) to 60% (w/w) of Q_{RMT} used in this cycle.
Step 2.3—stirring, settling, and decanting the contents at the end of Step 2.2 in the following stages:
   stage 2.3a. charging to said reaction vessel a suitable reaction medium, denoted as third settling reaction medium, at a predetermined second stirring temperature and a predetermined second stirring pH at a predetermined second stirring time;
   stage 2.3b. stirring and continuing to stir the mixture of stage 2.3a by maintaining the mixture at a predetermined second stirring continuation temperature, a predetermined second stirring continuation pH for a predetermined second stirring continuation time;
   stage 2.3c. stopping the stirring action and allowing the mixture of stage 2.3b to settle at a predetermined third settling pH, a predetermined third settling temperature for a predetermined third settling time; and
   stage 2.3d. decanting the liquid layer collected at the end of stage 2.3c near the top of said reaction vessel, the liquid layer denoted as Stream E, at a predetermined third decantation temperature, a predetermined third decantation pH and at a predetermined third decantation time; said Stream E being charged to a washings storage tank;
   wherein the values each of said second stirring temperature, said second stirring maintenance temperature, said third settling temperature, and said third decantation temperature are in the range of 0° C. and 200° C., preferably between 0° C. to 100° C.; the values of each of said second stirring pH, said second stirring maintenance pH, said third settling pH, and said third decantation pH are in the range of between 3 to 14, preferably between 4 to 12; the values of each of said second stirring time, said second stirring maintenance time, said third settling time, and said third decantation time are in the range of 5 minutes to 5 hours; preferably 30 minutes to 3 hours; and
   wherein the quantity of the reaction medium used in Step 2.3, denoted as Q_{RM2.3} is variable in the range of 0% (w/w) to 60% (w/w) of Q_{RMT} used in this cycle.
Step 2.4—separating and washing the solids obtained at the end of step 2.3, said separating and washing comprises the following stages:
   stage 2.4a. charging to said first reaction vessel a suitable reaction medium, denoted as first separation and washing reaction medium; wherein the quantity of said first separation and washing reaction medium, denoted as Q_{RM2.4}, is variable in the range of 0% (w/w) to 60% (w/w) of the Q_{RMT} used in this cycle.
   stage 2.4b. stirring and continuing to stir the mixture obtained at the end of stage 2.4a at a predetermined separation temperature in the range of 0° C. and 200° C., a predetermined separation pH in the range of between 3 to 14, preferably between 4 to 12; and a predetermined separation time in the range of 5 minutes to 5 hours; preferably 30 minutes to 3 hours;
   stage 2.4c. stopping the stirring action and separating solids and liquids from the mixture of solids and liquid obtained at the end of stage 2.4b by any of commonly known methods; and
   stage 2.4d charging the liquid stream obtained at the end of stage 2.4c as a result of the solid-liquid separation activity to said washings storage tank; is denoted as Stream F
Step 2.5—separating amino compounds by a method comprising the following stages:
   stage 2.5a. charging said Stream A of Step 2.1 and said Stream B of Step 2.2, either individually or in any combination, to a second reaction vessel equipped with an agitator and other attachments known to person skilled in the art;
   stage 2.5b. stirring the mixture obtained at the end of stage 2.5a at a second separation temperature that is in the range between 0° C. and 200° C., preferably in the range between 0° C. and 100° C., a second separation pH that is in the range between 3 to 14, preferably between 4 to 12; and for a second separation time that is in the range between 5 minutes to 5 hours; preferably 30 minutes to 3 hours; and
   stage 2.5c. stopping the stirring action and separating the amino compounds formed during the earlier steps of the current cycle by reducing the temperature of the reaction mixture to a predetermined third separation temperature in accordance with a predetermined cooling regime; preferable cooling regime being such that the period over which the temperature reduction is carried out is in the range between 5 minutes to 10 hours, more preferably between 30 minutes to 3 hours; and wherein the third separation temperature is in the range between 20° C. to −20° C., more preferably between 0° C. to −10° C.;
   stage 2.5d. maintaining the mixture obtained at the end of stage 2.5c at said second separation temperature for a cooling time;
Step 2.6—isolating the total mass obtained at the end of Step 2.5, the process of isolation comprising the steps of:
   stage 2.6a. isolating the total mass obtained at the end of Step 2.5 by any method known to a person skilled in the art; the liquid layer generated at the end of step 2.6a, denoted as Stream C, is collected in a mother liquor storage tank; and using required amounts of liquid from said mother liquor storage tank as stream D in all further cycles as necessary,
   stage 2.6b. washing the isolated mass obtained at the end of stage 2.6a using a suitable reaction medium, denoted as washing reaction medium; and
   stage 2.6c. charging the filtrate and washings obtained as a result of stages 2.6b and 2.6c to said washings storage tank.
4. A process as described in item 3, wherein in the first cycle of said green reaction sequence, a fresh reaction medium is used as the reaction medium in any or all of Steps 1.1, 1.2, 1.3, 1.4, 2.1, 2.2, 2.3, 2.4, and 2.6, that is said fresh reaction medium is used as any or all, or any combination thereof, of said start-up reaction medium, said reduction reaction medium, said neutralization reaction medium, said purification reaction medium, said first settling reaction medium, said second settling reaction medium, said third settling reaction medium, said first separation and washing reaction medium, and washing reaction medium.
5. A process as described in any of items 3 and 4, wherein for any cycle following the first cycle a fresh portion of the liquid stored in said washings storage tank is used as the reaction medium in any of Steps 1.1, 1.2, 1.3, 1.4, 2.1, and 2.2, that is the fresh portion of the liquid stored in said washings storage tank is used as any or all, or any combination thereof, of said start-up reaction medium, said reduction reaction medium, said neutralization reaction medium, said purification reaction medium, said first settling reaction medium, said second settling reaction medium.
6. A process as described in any of items 3 to 5, wherein the temperature in any or all of the stages 1.1b, 1.1c, 1.2c, 1.2d, 1.3b, 1.3c, 1.4b, 1.4c, or a combination thereof, is in the range of 50° C. to 100° C.
7. A process as described in any of items 3 to 6, wherein said first suitable acid and said second suitable acid are sulphuric acid.
8. A process as described in any of items 3 to 7, wherein the reducing agents of steps 1.1 and 1.2, namely said RA_{1.1} and RA_{1.2}, are G-CAT or any other reduction agent.
9. A process as described in any of items 3 to 8, wherein the neutralisation agent of Step 1.3 is R-Cat or any combination thereof, or any other proprietary neutralisation agent used on its own or in any combination with a combination of R-Cat and or G-Cat.
10. A process as described in any of items 3 to 9, wherein the nitro compound to be reduced is added to the step 1.2 in its entire quantity or in batches of any size at any interval.
11. A process as described in any of items 3 to 10, wherein the individual stages of step 1.2 are carried out in any sequence.
12. A process as described in any of items 3 to 11, wherein a salt of iron with inorganic or organic acids, preferably selected from a group of salts comprising ferrous sulphate, ferrous chloride, ferrous ammonium sulphate, ferrous oxalate, ferrous citrate, or any combination thereof, or a salt selected from a group comprising ammonium chloride, ammonium sulphate, other such salts, or any combination thereof, is used in place of said first suitable acid of step 1.1b.
13. A process as described in any of items 3 to 12, wherein said neutralising agent of stage 1.3b is selected from a group comprising hydroxides, carbonates, or bicarbonates of alkali metals, either individually or in any combination thereof; said hydroxides preferably being sodium hydroxide, potassium hydroxide, calcium hydroxide, lithium hydroxide; said carbonates preferably being sodium carbonate, potassium carbonate, calcium carbonate, or lithium carbonate; said bicarbonates preferably being sodium bicarbonate, potassium bicarbonate, lithium bicarbonate.

While the above description contains many specificities, these should not be construed as limitation in re scope of the invention, but rather as an exemplification of the preferred embodiments thereof. Many other variations are possible. Accordingly, the scope of the invention should be determined not by the embodiments illustrated, but by the appended claims and their legal equivalents.

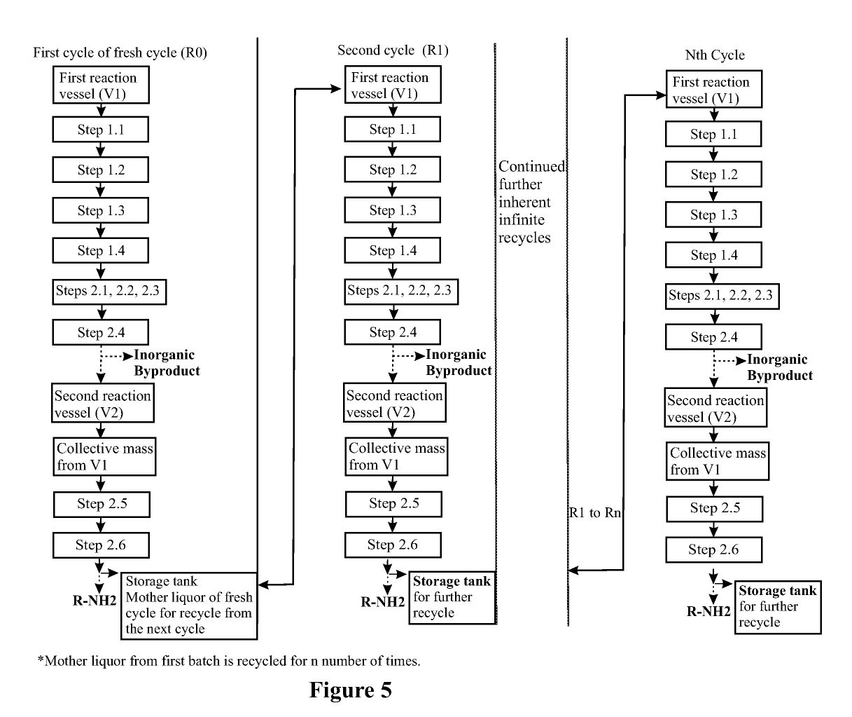

1. A sustainable chemical process of reduction of nitro-compounds, R—NO₂, or nitroso compounds, R—NO, into corresponding amino-compounds, R—NH₂, comprising a plurality of cycles, each of said cycles comprising a reaction sequence and an isolation sequence, characterised in that said nitroso or nitro compounds have a Sulphonic or carboxylic group, and that isolation sequence followed by said reaction sequence, are carried out in a steady state closed loop circuit capable of inherently and intrinsically recycling at source level all mother liquor generated, as shown FIG. 5.

2. A sustainable chemical process as claimed in claim 1 further characterized in that said plurality of cycles is greater than 25.

3. A sustainable chemical process as claimed in claim 1 further characterized in that said plurality of cycles is greater than 100.

4. A process as claimed in claim 3
wherein said green reaction sequence of a typical said cycle comprises the following steps:
step 1.1: creating start-up conditions for the reduction process, said step 1.1 further comprising the following stages:
charging a suitable reaction medium to a first reaction vessel, characterized in that for all cycles following the initial cycle said reaction medium is selected from Stream D, which has been identified in stage 2.6a of any cycle
optionally charging to said first reaction vessel a first suitable acid and agitating the mixture
adding to said first reaction vessel, upon completion of the agitation stage of stage 1.1c, a reducing agent RA_{1.1} in suitable quantity which is denoted as Q_{RA1.1};
step 1.2: reducing the nitro or nitroso compound to be reduced, wherein said step 1.2 further comprises the following stages:
a. adding R—NO₂ or R—NO, respectively a nitro or nitroso compound to be reduced, to said reaction vessel
b. charging a suitable reaction medium, denoted as reduction reaction medium, in suitable quantity to said first reaction vessel, characterized in that for all following cycles said reduction reaction medium is taken from Stream D
c. optionally adding to said first reaction vessel a second suitable acid for pH adjustment
d. adding a reducing agent, RA_{1.2}, to said first reaction vessel, either simultaneously with the R—NO₂ or R—NO compound to be reduced in stage 1.2a, or after the addition of acid of stage 1.2c;
Step 1.3: neutralizing the reaction mixture obtained at the end of Step 1.2, wherein said neutralization is carried out in the following stages:
a. optionally adding a suitable reaction medium, denoted as neutralization reaction medium, in a suitable quantity to said reaction vessel, characterized in that for all following cycles said neutralization reaction medium is taken from Stream D
b. adding to the reaction mixture obtained at the end of step 1.3a in said first reaction vessel a neutralizing agent, NA_{1.3},
c. allowing the neutralization of the mixture in said reaction vessel
Step 1.4—isolating the appropriate part of the contents of the said first reaction vessel obtained at the end of Step 1.3, wherein the isolation process comprises the following stages:
a. charging to the reaction mixture obtained at the end of stage 1.3c, a catalytic agent, preferably G-Cat, to form an isolation mixture
b. optionally adding a suitable reaction medium, denoted as isolation reaction medium, characterised in that for all following cycles said isolation reaction medium is taken from Stream D
c. maintaining the isolation mixture obtained at the end of stage 1.4b at a temperature between 0° C. and 200° C.
whereby a single cycle of said green reaction sequence is completed, and where after a cycle of green isolation sequence is carried out, said green isolation sequence comprising the following steps:
Step 2.1—settling and decanting the contents of said reaction vessel obtained at the end of said step 1.4, wherein said settling and decantation comprises following stages:
stage 2.1a. optionally charging a suitable reaction medium, denoted as first settling reaction medium, to the reaction mixture obtained at the end of step 1.4, characterised in that for all following cycles said first settling reaction medium is taken from Stream D
stage 2.1b. allowing the reaction mixture obtained at the end of stage 2.1b to settle down for a first settling time;
stage 2.1c. decanting the liquid layer formed at the end of stage 2.1b at a first decanting temperature and charging the decanted liquid denoted as Stream A, characterized in that Stream A is charged to Step 2.5 of same cycle or any of the following cycles;
Step 2.2—stirring, settling, and decanting the contents obtained at the end of Step 2.1, the stirring, settling and decanting comprising the following stages:
stage 2.2a. charging to said reaction vessel a suitable reaction medium, denoted as second settling reaction medium, characterised in that for all following cycles said second settling reaction medium is taken from Stream D
stage 2.2b. stirring and continuing to stir the mixture of stage 2.2a
stage 2.2c. stopping the stirring action and allowing the mixture of stage 2.2b to settle
stage 2.2d. decanting the liquid layer collected at the end of stage 2.2c, the liquid layer denoted as Stream B, characterized in that said Stream B being charged to Step 2.5 of the same cycle or any of the following cycles;
Step 2.3—stirring, settling, and decanting the contents at the end of Step 2.2 in the following stages:
stage 2.3a. charging to said reaction vessel a suitable reaction medium, denoted as third settling reaction medium; characterised in that for all following cycles said third settling reaction medium is taken from Stream G
stage 2.3b. stirring and continuing to stir the mixture of stage 2.3a
stage 2.3c. stopping the stirring action and allowing the mixture of stage 2.3b to settle; and
stage 2.3d. decanting the liquid layer collected at the end of stage 2.3c near the top of said reaction vessel, the liquid layer denoted as Stream E, characterized in that said Stream E being charged to a washings storage tank for recycling in further cycles;
Step 2.4—separating and washing the solids obtained at the end of step 2.3, said separating and washing comprises the following stages:
stage 2.4a. charging to said first reaction vessel a suitable reaction medium, denoted as first separation and washing reaction medium; characterized in that for all following cycles said first separation and washing reaction medium is taken from Stream G
stage 2.4b. stirring and continuing to stir the mixture obtained at the end of stage 2.4a
stage 2.4c. stopping the stirring action and separating solids and liquids from the mixture of solids and liquid obtained at the end of stage 2.4b; and
stage 2.4d charging the liquid stream obtained at the end of stage 2.4c as a result of the solid-liquid separation activity to said washings storage tank; denoted as Stream F
Step 2.5—separating amino compounds by a method comprising the following stages:
stage 2.5a. charging an isolation reaction medium characterized in that said isolation reaction medium is taken from said Stream A of said Step 2.1 and said Stream B of Step 2.2, either individually or in any combination, to a second reaction vessel;
stage 2.5b. stirring the mixture obtained at the end of stage 2.5a; and
stage 2.5c. stopping the stirring action and separating the amino compounds formed during the earlier steps of the current cycle;
stage 2.5d. maintaining the mixture obtained at the end of stage 2.5c at said second separation temperature for a cooling time;
Step 2.6—isolating the total mass obtained at the end of Step 2.5, the process of isolation comprising the steps of:
stage 2.6a. isolating the total mass obtained at the end of Step 2.5 by any method known to a person skilled in the art and collecting the liquid layer generated at the end of step 2.6a in a mother liquor storage tank, denoted as Stream C; and using required amounts of liquid from said mother liquor storage tank as Stream D in all further cycles as necessary,
stage 2.6b. washing the isolated mass obtained at the end of stage 2.6a using a suitable reaction medium, denoted as washing reaction medium, characterized in that said washing reaction medium is selected from fresh reaction medium stream and
stage 2.6c. charging the filtrate and washings obtained as a result of stages 2.6b and 2.6c to said washings storage tank.

5. A process as claimed in claim 4 further wherein
in stage 1.1a the quantity of the reaction medium used is denoted as Q_{RM1.1}, and is variable the range of 0% (w/w) to 40% (w/w) of Q_{RMT} used in this cycle;
wherein said Q_{RMT} is the total quantity of the reaction medium used in this cycle, said Q_{RMT} being determined such that the ratio, denoted as (Weight Ratio)_{RM}, of the weight of said Q_{RMT}, W_{RM}, to the weight of total amount of R—NO₂ or R—NO to be reduced in that single cycle W_{N}; the relationship between (Weight Ratio)_{RM}, W_{RM}, and W_{N} being represented by the equation
(Weight Ratio)_{RM}=W_{RM}/W_{N};
and wherein said (Weight Ratio)_{RM} is preferably in the range of 5 to 100, the more preferable range being 10 to 75, and;
in stage 1.1b the amount of acid charged is such that the pH of said start up reaction mixture is in the range between 1 to 9 and the temperature of the start-up mixture is in the range between 0° C. to 200° C.;
in stage 1.1c the mixture thus formed is agitated for a duration in the range of 0 minutes to 5 hours, more preferably between 0.5 hours to 2.5 hours, while maintaining the pH of the mixture during the agitation stage of stage 1.1c in the range between 1 to 9 while maintaining the temperature of the mixture during the agitation stage of stage 1.1c in the range between 0° C. to 200° C.; and
in stage 1.1d adding to said first reaction vessel, upon completion of the agitation stage of stage 1.1c, said reducing agent in a quantity denoted as Q_{RA1.1}, said Q_{RA1.1} being variable in the range of 0% to 100% of Q_{RAT};
wherein said Q_{RAT} is the total quantity of the reducing agent used in this cycle; said Q_{RAT} being determined such that the ratio, denoted as (Weight Ratio)_{RA}, of the weight of said Q_{RAT}, W_{RA}, to the weight of total amount of R—NO₂ or R—NO to be reduced in that single cycle, W_{N}; wherein the relationship between (Weight Ratio)_{RA}, W_{RA}, and W_{N} is represented by the equation:
(Weight Ratio)_{RA}=W_{RA}/W_{N};
and wherein said (Weight Ratio)_{RA} is preferably in the range of 0.25 to 25, the more preferable range being 0.5 to 2.5;
wherein said reducing agent is added either in its full required quantity, Q_{RA1.1}, or in batches, or continuously, or as any combination of these methods of addition, over a period of 0 minutes to 5 hours, preferably 0.5 hours to 2.5 hours; and wherein the pH of the mixture in said first reaction vessel at the time of addition of said reducing agent is between 1 to 9, preferably between 2 to 7, more preferably between 4 to 6;
in stage 1.2a reduction period is in the range of 0 to 25 hours;
in stage 1.2c the acid added is in a quantity to bring the pH value of the mixture thus formed within the range between 1 to 9, while maintaining the temperature of the mixture formed by addition the acid to said reduction mixture between 0° C. to 200° C.; and
in stage 1.2d said RA_{1.2} is added either simultaneously with the R—NO₂ or R—NO compound to be reduced in stage 1.2a, or after the addition of acid of stage 1.2c, thereby forming a reduction agent mixture; wherein said RA_{1.2} is added at a reduction time such that the pH of said reduction agent mixture is in a range between 1 to 9 and such that the temperature of said reduction mixture is in the range between 0° C. to 200° C.;
wherein the quantity of RA_{1.2}, denoted as Q_{R1.2}, is such that said Q_{R1.2} is the difference between Q_{RT} and Q_{R1.1};
in stage 1.3a the quantity of the reaction medium used in step 1.3, denoted as Q_{RM1.3}, is variable in the range of 0% (w/w) to 40% (w/w) of Q_{RMT};
in stage 1.3b the quantity of said neutralizing agent used, Q_{NAT}, is the total quantity of the neutralizing agent to be used in this cycle; said Q_{NAT} being determined such that the ratio, denoted as (Weight Ratio)_{NA}, of the weight of said Q_{NAT}, W_{NA}, to the weight of total amount of R—NO₂ or R—NO to be reduced in that single cycle, W_{N}; wherein the relationship between (Weight Ratio)_{NA}, W_{NA}, and W_{N} is represented by the equation:
(Weight Ratio)_{NA}=W_{NA}/W_{N};
and wherein said (Weight Ratio)_{NA} is preferably in the range of 0 to 2.5, the more preferable range being 0.05 to 0.25; and
wherein said neutralizing agent is added over a period in the range between 0 minutes to 5 hours, more preferably between 0.5 hours to 2.5 hours, at a temperature in the range between 0° C. to 200° C., and at a pH in the range between 1 to 9, preferably 2 to 8; more preferably 5.5 to 7.5;
wherein R-Cat or G-Cat is the preferred neutralizing agent; and
in stage 1.3c the neutralization takes place at a temperature between 0° C. to 200° C., at a pH between 1 to 9 carried out over a period between 0 hours to 10 hours;
wherein the contents of said first reaction vessel during any or all stages of 1.3a to 1.3c are optionally stirred for any duration of the individual stages using said agitator rotating at a rate between 0 to 500 RPM;
in stage 1.4a the quantity of said R-Cat or G-Cat being such that its weight ratio with R—NO₂ or R—NO is in the range of a 0.05 w/w to 5 w/w, added at a time such that the pH of said purification reaction mixture is in the range of 1 to 12;
in stage 1.4b the separation reaction mixture is added at a temperature between 0° C. and 200° C., and at pH level between the range of 1 to 12; and
in stage 1.4c the separation mixture obtained at the end of stage 1.4b is maintained at a temperature between 0° C. and 200° C., for a period in the range of 0 hours to 24 hours;
in stage 2.1a the first settling reaction mixture is charged while maintaining the temperature of the mixture in the range between 0° C. and 200° C., and the pH of the mixture in the range between 1 to 12, and wherein the quantity of said first settling reaction mixture used, denoted as Q_{RM2.1}, is variable in the range of 0% (w/w) to 60% (w/w) of the Q_{RMT} used in this cycle;
in stage 2.1b the temperature of the mixture in the range between 0° C. and 200° C., and the pH of the mixture in the range between 1 to 12 and said first settling time is in the range between 1 minute to 10 hours;
in stage 2.1c the first decanting temperature is in the range between 0° C. and 200° C., a first decanting pH in the range between 1 to 12, and first decanting time in the range between 1 minute to 10 hours, the decanting the liquid layer collected at the end of stage 2.1c, denoted as Stream A;
in stage 2.2a the second settling reaction mixture is added at a predetermined first stirring temperature and a predetermined first stirring pH at a predetermined first stirring time;
in stage 2.2b stirring and continuing to stir the mixture of stage 2.2a by maintaining the mixture at a predetermined first stirring continuation temperature, a predetermined first stirring continuation pH for a predetermined first stirring continuation time;
in stage 2.2c stopping the stirring action and allowing the mixture of stage 2.2b to settle at a predetermined second settling pH, a predetermined second settling temperature for a predetermined second settling time; and
in stage 2.2d decanting the liquid layer collected at the end of stage 2.2c, the liquid layer denoted as Stream B, at a predetermined second decantation temperature, a predetermined second decantation pH and at a predetermined second decantation time; said Stream B being charged to step 2.5 of the same cycle or any of the following cycles;
wherein the values each of said first stirring temperature, said first stirring continuation temperature, and said second decantation temperature are in the range of 0° C. and 200° C.; the values of each of said first stirring pH, said first stirring continuation pH, and said second decantation pH are in the range of 1 to 12; the values of each of said first stirring time, said first stirring continuation time, and said second decantation time are in the range of 5 minutes to 5 hours; and
wherein the quantity of the reaction medium used in step 2.2, denoted as Q_{RM2.2} is variable in the range of 0% (w/w) to 60% (w/w) of Q_{RMT} used in this cycle;
in stage 2.3a the third settling reaction mixture is added at a predetermined second stirring temperature and a predetermined second stirring pH at a predetermined second stirring time;
in stage 2.3b stirring and continuing to stir the mixture of stage 2.3a by maintaining the mixture at a predetermined second stirring continuation temperature, a predetermined second stirring continuation pH for a predetermined second stirring continuation time;
stage 2.3c stopping the stirring action and allowing the mixture of stage 2.3b to settle at a predetermined third settling pH, a predetermined third settling temperature for a predetermined third settling time; and
stage 2.3d decanting the liquid layer collected at the end of stage 2.3c near the top of said reaction vessel, the liquid layer denoted as Stream E, at a predetermined third decantation temperature, a predetermined third decantation pH and at a predetermined third decantation time; said Stream E being charged to a washings storage tank;
wherein the values each of said second stirring temperature, said second stirring maintenance temperature, said third settling temperature, and said third decantation temperature are in the range of 0° C. and 200° C.; the values of each of said second stirring pH, said second stirring maintenance pH, said third settling pH, and said third decantation pH are in the range of 1 to 12; the values of each of said second stirring time, said second stirring maintenance time, said third settling time, and said third decantation time are in the range of 5 minutes to 5 hours; and
wherein the quantity of the reaction medium used in step 2.3, denoted as Q_{RM2.3} is variable in the range of 0% (w/w) to 60% (w/w) of Q_{RMT} used in this cycle;
in stage 2.4a the quantity of said first separation and washing reaction mixture, denoted as Q_{RM2.4}, is variable in the range of 0% (w/w) to 60% (w/w) of the Q_{RMT} used in this cycle.
in stage 2.4b stirring and continuing to stir the mixture obtained at the end of stage 2.4a at a predetermined separation temperature in the range of 0° C. and 200° C., a predetermined separation pH in the range of 1 to 12, and a predetermined separation time in the range of 5 minutes to 5 hours, the separated liquid at the end of stage 2.4b is denoted as Stream F;
in stage 2.5b stirring the mixture obtained at the end of stage 2.5a at a second separation temperature that is in the range between 0° C. and 200° C., a second separation pH that is in the range between 1 to 12, and for a second separation time that is in the range between 5 minutes to 5 hours; and
in stage 2.5c stopping the stirring action and separating the amino compounds formed during the earlier steps of the current cycle by reducing the temperature of the reaction mixture to a predetermined third separation temperature in accordance with a predetermined cooling regime; preferable cooling regime being such that the period over which the temperature reduction is carried out is in the range between 5 minutes to 10 hours; and wherein the third separation temperature is in the range between 20° C. to −20° C.

6. A process as claimed in claim 5 wherein
in stage 1.1b the pH of the startup reaction mixture is preferably between 4 to 6;
in stage 1.1c the agitation is preferably carried out between 0.5 hours to 2.5 hours, while maintaining the pH of the mixture during the agitation stage between 3 to 7;
in stage 1.1d said reducing agent is added over a period preferably between 0.5 hours to 2.5 hours; and wherein the pH of the mixture at the time of addition of said reducing agent is preferably between 2 to 7;
in stage 1.2c the reduction mixture has a pH preferably between 4 to 6; and
in stage 1.2d the reducing agent is added at a reduction time such that the pH of said reduction agent mixture is in a range preferably between 4 to 6;
in stage 1.3a optionally adding a suitable reaction medium, denoted as neutralization RM, in a suitable quantity to said reaction vessel, wherein quantity of the reaction medium used in step 1.3, denoted as Q_{RM1.3}, is variable in the range of 0% (w/w) to 40% (w/w) of Q_{RMT};
in stage 1.3b said neutralizing agent is added over a period preferably between 0.5 hours to 2.5 hours, at a pH preferably between 2 to 8;
in stage 1.3c the neutralization takes place at a pH preferably between 2 to 8, said neutralization being carried out over a period preferably in the range of 30 minutes to 5 hours;
in stage 1.4a the R-Cat or G-Cat is charged in weight ratio with R—NO₂ or R—NO is in the preferable range of 0.5 w/w to 2.5 w/w, at a time such that the pH of said purification reaction mixture is in the range preferably between 4 to 11;
in stage 1.4b the separation reaction medium is added at pH level preferably between 4 to 11; and
in stage 1.4c the separation mixture is maintained at a temperature preferably between 0° C. to 100° C., for a period preferably in the range of 30 minutes to 5 hours;
in stage 2.1b the reaction mixture obtained at the end of stage 2.1b is allowed to settle down at the pH preferably between 4 to 7; wherein said first settling time is preferably between 30 minutes to 3 hours;
in stage 2.1c said first decanting pH is preferably between 4 to 11 and first decanting time preferably between 30 minutes to 3 hours;
in stage 2.2b the values each of said first stirring temperature, said first stirring continuation temperature, and said second decantation temperature respectively are preferably between 0° C. to 100° C.; the values of each of said first stirring pH, said first stirring continuation pH, and said second decantation pH are in the range preferably between 4 to 11; the values of each of said first stirring time, said first stirring continuation time, and said second decantation time are preferably between 30 minutes to 3 hours; and
in stage 2.3d the values each of said second stirring temperature, said second stirring maintenance temperature, said third settling temperature, and said third decantation temperature are preferably between 0° C. to 100° C.; the values of each of said second stirring pH, said second stirring maintenance pH, said third settling pH, and said third decantation pH are in the range preferably between 4 to 11; the values of each of said second stirring time, said second stirring maintenance time, said third settling time, and said third decantation time are in the range of preferably 30 minutes to 3 hours; and
in stage 2.4b the separation pH is in the range preferably between 4 to 11, and a predetermined separation time in the range preferably 30 minutes to 3 hours;
in stage 2.5b stirring the mixture obtained at the end of stage 2.5a at a second separation pH that is in the range preferably between 4 to 9, and for a second separation time that is in the range between preferably 30 minutes to 3 hours; and
in stage 2.5c the period over which the temperature reduction is carried out is in the range preferably between 30 minutes to 3 hours; and wherein the third separation temperature is in the range preferably between 0° C. to −10° C.

7. A process as claimed in claim 6, wherein said first suitable acid and said second suitable acid are sulphuric acid.

8. A process as claimed in claim 7, wherein the reducing agents of steps 1.1 and 1.2, namely said RA_{1.1} and RA_{1.2}, is suitable reduction agent.

9. A sustainable chemical process of nitro-compounds, R—NO₂, or nitroso compounds, R—NO, into corresponding amino-compounds, R—NH₂, as claimed in claim 8, wherein the neutralisation agent of step 1.3 is any proprietary neutralisation agent.

10. A process as claimed in claim 9, wherein the nitro compound to be reduced is added to the step 1.2 in its entire quantity or in batches of any size at any interval.

11. A process as claimed in claim 10, wherein said neutralising agent of stage 1.3b is selected from a group comprising hydroxides, carbonates, or bicarbonates of alkali metals, either individually or in any combination thereof; said hydroxides preferably being sodium hydroxide, potassium hydroxide, calcium hydroxide, lithium hydroxide; said carbonates preferably being sodium carbonate, potassium carbonate, calcium carbonate, or lithium carbonate; said bicarbonates preferably being sodium bicarbonate, potassium bicarbonate, lithium bicarbonate; and wherein said reducing agent of step 1.1d comprises multifunctional, chemical reduction formulation selected from a group comprising fine iron powder, electrolyte salt of various metals such as sodium, magnesium, calcium, iron, nickel, cobalt, tin, zinc, titanium, copper, manganese, and any other metals with multiple valancies, customized grade of activated carbon, and specialty additives like polyelectrolytes, anti foaming agents, dispersing agents, anti oxidants, emulsifying agents, mass transfer enhancing agents, anti caking agents, UV stabilizers, solubilising agents, preferably fine iron powder.

12. A process as claimed in claim 11, wherein the neutralising agents are selected from a group comprising agent in the form of hydroxides of alkali metals like sodium hydroxide, potassium hydroxide, calcium hydroxide, lithium hydroxide, carbonates or bicarbonates of alkali metals like sodium carbonate, sodium bicarbonate, potassium carbonate, potassium bicarbonate, calcium carbonate, lithium carbonate, other such salts or any combination thereof.

13. A process as claimed in claim 12 wherein said number of cycles is greater than 100.
